(19) United States
(12) Patent Application Publication
(10) Pub. No.: US 2021/0308183 A1
(43) Pub. Date: Oct. 7, 2021

(54) CHIMERIC ANTIGEN RECEPTOR T CELLS DERIVED FROM IMMUNOENGINEERED PLURIPOTENT STEM CELLS

(71) Applicant: The Regents of the University of California, Oakland, CA (US)

(72) Inventors: Sonja Schrepfer, Burlingame, CA (US); Tobias Deuse, Burlingame, CA (US)

(73) Assignee: The Regents of the University of California, Oakland, CA (US)

(21) Appl. No.: 17/260,222

(22) PCT Filed: Jul. 17, 2019
(86) PCT No.: PCT/US19/42123

§ 371 Date: Jan. 14, 2021

Related U.S. Application Data
(60) Provisional application No. 62/698,941, filed on Jul. 17, 2018.

(51) Int. Cl. A61K 35/17; C12N 15/11; C12N 9/22; C12N 5/0783

(52) U.S. Cl. CPC A61K 35/17; C12N 15/111; A61K 38/00; C12N 5/0636; C12N 9/22

(57) ABSTRACT

The invention provides universally acceptable “off-the-shelf” hypoimmune pluripotent (HIP) cells and hypoimmune chimeric antigen receptor T (CAR-T) cells derived from the HIP cells. The engineered therapeutic cells can be administered to subjects as an adoptive cell-based immunotherapy to treat cancer.

### I. CROSS REFERENCE TO RELATED APPLICATIONS

This application claims priority to 62/698,941 filed on Jul. 17, 2018, incorporated by reference herein in its entirety.

### II. FIELD OF THE INVENTION

The present invention relates to the field of adoptive immunotherapy. The invention provides chimeric antigen receptor (CAR) expressing immune cells, e.g., T cells that have been differentiated from hypoimmunogenic pluripotent (HIP) stem cells comprising a nucleic acid encoding the CAR. The engineered HIP cells are genetically modified to be homozygous null for the beta-2 microglobulin (B2M) gene, homozygous null for the class II transactivator (CIITA) gene, and to overexpress CD47.

### III. BACKGROUND OF THE INVENTION

Adoptive cell immunotherapy utilizes antigen-specific immune cells, e.g., T cell or natural killer (NK) cells, to treat a number of diseases including cancer and antibody-mediated transplant rejection. Unfortunately, current adoptive T cell therapies are limited by the lack of universal tumor-specific T cells. For instance, Kymriah™ (tisagenlecleucel, Novartis) and Yescarta™ (axicabtagene ciloleucel, Kite) uses a patient's own T cells to produce the CAR-T therapy.

Such adoptive T cell therapies are based on autologous cell transfer. T lymphocytes are recovered from a patient, genetically modified or selected ex vivo, cultivated in vitro in order to amplify the number of cells, and finally infused into the patient. In addition to lymphocyte infusion, the patient may also be pre-conditioned with radiation or chemotherapy and administration of lymphocyte growth factors such as IL-2 to promote and support engraftment of the T cells and/or a therapeutic response

Each patient receives an individually manufactured treatment, using the patient's own lymphocytes. Such autologous therapies face substantial technical and logistic problems. For instance, the therapeutic cells must be generated in expensive dedicated facilities staffed with expert personnel and they must be generated in a short time following a patient's diagnosis. In some cases, due to pretreatment of the patient the isolated lymphocytes may be poorly functional and present in very low numbers, thus making it challenging to produce an effective amount of therapeutic cells for treating the patient.

Therefore, there is a need for “off-the-shelf” therapeutic antigen-specific T cells for use in adoptive immunotherapies.

### IV. SUMMARY OF THE INVENTION

In one aspect, the present invention provides an isolated hypoimmunogenic or hypoimmune pluripotent stem cell (HIP cell) comprising a nucleic acid encoding a chimeric antigen receptor (CAR), wherein endogenous β-2 microglobulin (B2M) gene activity and endogenous class II transactivator (CIITA) gene activity have been eliminated and CD47 expression has been increased. The CAR can comprise an extracellular domain, a transmembrane domain, and an intracellular signaling domain.

In some embodiments, the extracellular domain binds to an antigen selected from the group consisting of CD19, CD20, CD22, CD38, CD123, CS1, CD171, BCMA, MUC16, ROR1, and WT1. In certain embodiments, the extracellular domain comprises a single chain variable fragment (scFv). In some embodiments, the transmembrane domain comprises CD3ζ, CD4, CD8α, CD28, 4-1BB, OX40, ICOS, CTLA-4, PD-1, LAG-3, and BTLA. In certain embodiments, the intracellular signaling domain comprises CD3ζ, CD28, 4-1BB, OX40, ICOS, CTLA-4, PD-1, LAG-3, and BTLA.

In certain embodiments, the CAR comprises an anti-CD19 scFv domain, a CD28 transmembrane domain, and a CD3 zeta signaling intracellular domain. In some embodiments, the CAR comprises anti-CD19 scFv domain, a CD28 transmembrane domain, a 4-1BB signaling intracellular domain, and a CD3 zeta signaling intracellular domain.

In various embodiments, the nucleic acid encoding the CAR is introduced into the HIP cell after B2M gene activity and CIITA gene have been eliminated and CD47 expression has been increased.

In particular embodiments, the human HIP cell is a human engineered induced pluripotent stem cell (human engineered iPSC), the B2M gene is human B2M gene, the CIITA gene is human B2M gene, and the increased CD47 expression results from introducing into the human engineered iPSC at least one copy of a human CD47 gene under the control of a promoter. In other embodiments, the mouse HIP cell is a mouse engineered iPSC, the B2M gene is mouse B2M gene, the CIITA gene is mouse B2M gene, and the increased CD47 expression results from introducing into the mouse engineered iPSC at least one copy of a mouse CD47 gene under the control of a promoter. The promoter can be a constitutive promoter.

In some embodiments, elimination of B2M gene activity results from a Clustered Regularly Interspaced Short Palindromic Repeats (CRISPR)/Cas9 reaction that disrupts both alleles of the B2M gene. In certain embodiments, elimination of CIITA gene activity results from a CRISPR/Cas9 reaction that disrupts both alleles of the CIITA gene.

In some embodiments, the suicide gene is a herpes simplex virus thymidine kinase (HSV-tk) gene and the trigger agent is ganciclovir. In some instances, the HSV-tk gene encodes a protein comprising at least 90% sequence identity to SEQ ID NO:4. In certain instances, the HSV-tk gene encodes a protein comprising the amino acid sequence of SEQ ID NO:4.

In certain embodiments, the suicide gene is an Escherichia coli cytosine deaminase (CD) gene and the trigger agent is 5-fluorocytosine (5-FC). The CD gene can encode a protein comprising at least 90% sequence identity to SEQ ID NO:5. In some cases, the CD gene encodes a protein comprising the amino acid sequence of SEQ ID NO:5.

In various embodiments, the suicide gene encodes an inducible caspase 9 protein and the trigger agent is a chemical inducer of dimerization (CID). In certain instances, the inducible caspase 9 protein comprises at least 90% sequence identity to SEQ ID NO:6. In other instances, the inducible caspase 9 protein comprises the amino acid sequence of SEQ ID NO:6.

In another aspect of the invention, provided is an isolated hypoimmune CAR-T (HI-CAR-T) cell produced by in vitro differentiation of any one of the HIP cells described herein.

In some embodiments, the HI-CAR-T cell is a cytotoxic hypoimmune CAR-T cell.

In various embodiments, the in vitro differentiation comprises culturing the HIP cell carrying a CAR construct in a culture media comprising one or more growth factors or cytokines selected from the group consisting of bFGF, EPO, Flt3L, IGF, IL-3, IL-6, IL-15, GM-CSF, SCF, and VEGF. In some embodiments, the culture media further comprises one or more selected from the group consisting of a BMP activator, a GSK3 inhibitor, a ROCK inhibitor, a TGFβ receptor/ALK inhibitor, and a NOTCH activator.

In particular embodiments, isolated HI-CAR-T cell produced by in vitro differentiation of any one of the HIP carrying the CAR-T construct is for use as a treatment of cancer.

In another aspect of the invention, provided is a method of treating a patient with cancer by administering a composition comprising a therapeutically effective amount of any of the isolated HI-CAR-T cells described herein. In some embodiments, the composition further comprises a therapeutically effective carrier.

In some embodiments, the administration step comprises intravenous administration, subcutaneous administration, intranodal administration, intratumoral administration, intrathecal administration, intrapleural administration, and intraperitoneal administration. In certain instances, the administration further comprises a bolus or by continuous perfusion.

In some embodiments, the cancer is a blood cancer selected from the group consisting of leukemia, lymphoma, and myeloma. In various embodiments, the cancer is a solid tumor cancer or a liquid tumor cancer.

In another aspect, the present invention provides a pure population of HI-CAR-T cells derived from a population of isolated HIP cells carrying the CAR construct by a method comprising in vitro differentiation, wherein the isolated HIP cells comprise a nucleic acid encoding a chimeric antigen receptor (CAR) and a suicide gene that is activated by a trigger agent that can induce the HIP cells to die, and wherein endogenous β-2 microglobulin (B2M) gene activity and endogenous class II transactivator (CIITA) gene activity have been eliminated and CD47 expression has been increased in the HIP cells.

In some embodiments, the suicide gene is a herpes simplex virus thymidine kinase (HSV-tk) gene and the trigger agent is ganciclovir, the suicide gene is an Escherichia coli cytosine deaminase (CD) gene and the trigger agent is 5-fluorocytosine (5-FC), or the suicide gene is an inducible caspase 9 protein and the trigger agent is a chemical inducer of dimerization (CID).

In some embodiments, the HI-CAR-T cells are a cytotoxic hypoimmune CAR-T cells.

In some embodiments, the in vitro differentiation comprises culturing the HIP cells in a culture media comprising one or more growth factors or cytokines selected from the group consisting of bFGF, EPO, Flt3L, IGF, IL-3, IL-6, IL-15, GM-CSF, SCF, and VEGF. In some embodiments, the culture media further comprises one or more selected from the group consisting of a BMP activator, a GSK3 inhibitor, a ROCK inhibitor, a TGFβ receptor/ALK inhibitor, and a NOTCH activator. In some instances, the in vitro differentiation comprises culturing the HIP cells on feeder cells. In some embodiments, the feeder cells are endothelial cells. In certain embodiments, the feeder cells are endothelial cells derived from HIP cells, such as but not limited to human HIP cells. In some embodiments, the in vitro differentiation comprises culturing in simulated microgravity. In certain embodiments, the culturing in simulated microgravity is for at least 72 hours. In various embodiments, the method further comprises culturing the HI-CAR-T cells in a negative selection media comprising the trigger agent to induce the HIP cells to die, thereby producing a population of isolated HI-CAR-T cells that is substantially free or free of the HIP cells. Such isolated HI-CAR-T cells can be for use as a treatment of cancer.

In some embodiments, provided herein is a method of treating a patient with cancer by administering a composition comprising a therapeutically effective amount of any one of the pure population of isolated HI-CAR-T cells. The compositions can also include a therapeutically effective carrier.

In some embodiments, the administration step comprises intravenous administration, subcutaneous administration, intranodal administration, intratumoral administration, intrathecal administration, intrapleural administration, and intraperitoneal administration. In certain instances, the administration further comprises a bolus or by continuous perfusion.

In some embodiments, the cancer is a blood cancer selected from the group consisting of leukemia, lymphoma, and myeloma. In various embodiments, the cancer is a solid tumor cancer or a liquid tumor cancer.

In another aspect, the present invention provides a method of making any one of the isolated hypoimmune CAR-T cells (HI-CAR-T cells) described herein. The method includes in vitro differentiating of any one of the HIP cells of the invention wherein in vitro differentiating comprises culturing the HIP cell in a culture media comprising one or more growth factors or cytokines selected from the group consisting of bFGF, EPO, Flt3L, IGF, IL-2, IL-3, IL-6, IL-7, IL-15, GM-CSF, SCF, and VEGF. In some embodiments, the culture media further comprises one or more selected from the group consisting of a BMP activator, a GSK3 inhibitor, a ROCK inhibitor, a TGFβ receptor/ALK inhibitor, and a NOTCH activator.

In some embodiments, the in vitro differentiating comprises culturing the HIP cells on feeder cells. In various embodiments, the in vitro differentiating comprises culturing in simulated microgravity. In certain instances, the culturing in simulated microgravity is for at least 72 hours.

### V. BRIEF DESCRIPTION OF THE DRAWINGS

FIG. 1 shows Elispot results of mouse B2m−/−Ciita−/−CD47 tg iPSCs incubated with mouse natural killer (NK) cells (approximately 95% NK cells and 5% macrophages).

FIG. 2 shows Elispot results of human B2M−/−CIITA−/−CD47 tg iPSCs incubated with human NK cells (approximately 95% NK cells and 5% macrophages).

FIG. 3 shows Elispot results of mouse B2m−/−Ciita−/−CD47 tg iPSCs incubated with human NK cells (approximately 95% NK cells and 5% macrophages).

FIG. 4 shows Elispot results of human B2M−/−CIITA−/−CD47 tg iPSCs incubated with mouse NK cells (approximately 95% NK cells and 5% macrophages).

FIG. 5 shows phagocytosis assay results of firefly luciferase labeled human B2M−/−CIITA−/−CD47 tg iPSCs co-cultured with human macrophages.

FIG. 6 shows phagocytosis assay results of firefly luciferase labeled mouse B2m−/−Ciita−/−CD47 tg iPSCs co-cultured with mouse macrophages.

FIG. 7 shows phagocytosis assay results of firefly luciferase labeled human B2M−/−CIITA−/−CD47 tg iPSCs co-cultured with mouse macrophages.

FIG. 8 shows phagocytosis assay results of firefly luciferase labeled mouse B2m−/−Ciita−/−CD47 tg iPSCs co-cultured with human macrophages.

FIG. 9 shows differentiation of HIP cells described herein into T cells.

FIGS. 10A and 10B show differentiation of HIP cells into CD3+ cells, CD4+ cells, and CD8+ cells. FIG. 10A shows cells at day 23 (D23) of differentiation on OP9-DL1 cells. FIG. 10B shows cells at day 30 (D30) of differentiation off feeder cells and with CD3/CD28 stimulation.

FIG. 11 shows differentiation of HIP cells into T cells (e.g., CD3+ cells, CD4+ cells, and CD8+ cells) at day 23 (D23) of differentiation on feeder cells with CD3/CD28 stimulation.

FIG. 12 shows endothelial progenitor cells derived from HIP cells.

FIGS. 13A-13C show human HIP cells cultured with endothelial progenitor cells (EPCs) that were differentiated into CD4+ T cells (FIG. 13A), naïve CD4+ cells (CD45RA+CCR7+CD4+ cells; FIG. 13B), and central memory CD4+ T cells (CD45RA−CCR7+CD4+ cells; FIG. 13C). ** denotes p<0.001; unpaired student's t-test.

FIGS. 14A and 14B show human T cells derived from human HIP cells using simulated microgravity (sμg) for 72 hours. FIG. 14A shows the morphology of the human T cells derived from human HIP cells. FIG. 14B shows the cell viability of the human T cells. P=n.s.; unpaired student's t-test.

FIG. 15 shows human CD8+ T cells derived from human HIP cells using simulated microgravity (sμg) for 72 hours. * denotes p<0.05; unpaired student's t-test.

FIG. 16 shows human CD8+ T cells derived from human HIP cells using simulated microgravity (sμg) for 72 hours and 10 days.

FIG. 17 shows human CD8+CD45RA+CCR7+ T cells and human CD8+CD45RA+CCR7−T cells derived from human HIP cells using simulated microgravity (sμg) for 72 hours followed by treatment at 1 g for 72 hours. * denotes p<0.05; unpaired student's t-test.

FIG. 18 shows human CD8+ T cells derived from human HIP cells using simulated microgravity and cytokine stimulation.

### VI. DETAILED DESCRIPTION OF THE INVENTION

### A. Introduction

The invention provides HypoImmunogenic Pluripotent (“HIP”) cells that avoid host immune responses due to several genetic manipulations as outlined herein. The cells lack major immune antigens that trigger immune responses and are engineered to avoid phagocytosis. This allows the derivation of “off-the-shelf” cell products for generating specific tissues and organs. The benefit of being able to use human allogeneic HIP cell derivatives in human patients results in significant benefits, including the ability to avoid long-term adjunct immunosuppressive therapy and drug use generally seen in allogeneic transplantations. It also provides significant cost savings as cell therapies can be used without requiring individual treatments for each patient. Recently, it was shown that cell products generated from autologous cell sources may become subject to immune rejection with few or even one single antigenic mutation. Thus, autologous cell products are not inherently non-immunogenic. Also, cell engineering and quality control is very labor and cost intensive and autologous cells are not available for acute treatment options. Only allogeneic cell products will be able to be used for a bigger patient population if the immune hurdle can be overcome. HIP cells will serve as a universal cell source for the generation of universally-acceptable derivatives.

The present invention is directed to the exploitation of the fetomaternal tolerance that exists in pregnant women. Although half of a fetus' human leukocyte antigens (HLA) are paternally inherited and the fetus expresses major HLA mismatched antigens, the maternal immune system does not recognize the fetus as an allogeneic entity and does not initiate an immune response, e.g. as is seen in a “host versus graft” type of immune reaction. Fetomaternal tolerance is mainly mediated by syncytiotrophoblast cells in the fetal-maternal interface. Syncytiotrophoblast cells show little or no proteins of the major histocompatibility complexes I and II (MHC-I and MHC-II), as well as increased expression of CD47, known as the “don't eat me” protein that suppresses phagocytic innate immune surveillance and elimination of HLA-devoid cells. Surprisingly, the same tolerogenic mechanisms that prevent rejection of the fetus during pregnancy also allow the HIP cells of the invention to escape rejection and facilitate long-term survival and engraftment of these cells after allogeneic transplantation.

These results are additionally surprising in that this fetomaternal tolerance can be introduced with as little as three genetic modifications (as compared to the starting iPSCs, e.g. human iPSCs), two reductions in activity (“knock outs” as further described herein) and one increase in activity (a “knock in” as described herein). Generally, others of skill in the art have attempted to suppress immunogenicity of iPSCs but have been only partially successful; see Rong et al., Cell Stem Cell 14:121-130 (2014) and Gornalusse et al., Nature Biotech doi:10.1038/nbt.3860).

This application is related to International Application No. PCT/US18/13688, filed on Jan. 14, 2018 and U.S. Provisional Application No. 62/445,969, filed Jan. 13, 2017, the disclosures in their entirety are herein incorporated by reference, in particular, the examples, figures, figure descriptions, and descriptions of producing hypoimmunogenic pluripotent stem cells and differentiating such cells into other cell types.

Thus, the invention provides for the generation of HIP cells from pluripotent stem cells, and then their maintenance, differentiation and ultimately transplantation of their derivatives into patients in need thereof.

### B. Definitions

The term “pluripotent cells” refers to cells that can self-renew and proliferate while remaining in an undifferentiated state and that can, under the proper conditions, be induced to differentiate into specialized cell types. The term “pluripotent cells,” as used herein, encompass embryonic stem cells and other types of stem cells, including fetal, amnionic, or somatic stem cells. Exemplary human stem cell lines include the H9 human embryonic stem cell line. Additional exemplary stem cell lines include those made available through the National Institutes of Health Human Embryonic Stem Cell Registry and the Howard Hughes Medical Institute HUES collection (as described in Cowan, C. A. et. al, New England J. Med. 350:13. (2004), incorporated by reference herein in its entirety.)

“Pluripotent stem cells” as used herein have the potential to differentiate into any of the three germ layers: endoderm (e.g. the stomach linking, gastrointestinal tract, lungs, etc), mesoderm (e.g. muscle, bone, blood, urogenital tissue, etc) or ectoderm (e.g. epidermal tissues and nervous system tissues). The term “pluripotent stem cells,” as used herein, also encompasses “induced pluripotent stem cells”, or “iPSCs”, a type of pluripotent stem cell derived from a non-pluripotent cell. Examples of parent cells include somatic cells that have been reprogrammed to induce a pluripotent, undifferentiated phenotype by various means. Such “iPS” or “iPSC” cells can be created by inducing the expression of certain regulatory genes or by the exogenous application of certain proteins. Methods for the induction of iPS cells are known in the art and are further described below. (See, e.g., Zhou et al., Stem Cells 27 (11): 2667-74 (2009); Huangfu et al., Nature Biotechnol. 26 (7): 795 (2008); Woltjen et al., Nature 458 (7239): 766-770 (2009); and Zhou et al., Cell Stem Cell 8:381-384 (2009); each of which is incorporated by reference herein in their entirety.) The generation of induced pluripotent stem cells (iPSCs) is outlined below. As used herein, “hiPSCs” are human induced pluripotent stem cells, and “miPSCs” are murine induced pluripotent stem cells.

“Pluripotent stem cell characteristics” refer to characteristics of a cell that distinguish pluripotent stem cells from other cells. The ability to give rise to progeny that can undergo differentiation, under the appropriate conditions, into cell types that collectively demonstrate characteristics associated with cell lineages from all of the three germinal layers (endoderm, mesoderm, and ectoderm) is a pluripotent stem cell characteristic. Expression or non-expression of certain combinations of molecular markers are also pluripotent stem cell characteristics. For example, human pluripotent stem cells express at least several, and in some embodiments, all of the markers from the following non-limiting list: SSEA-3, SSEA-4, TRA-1-60, TRA-1-81, TRA-2-49/6E, ALP, Sox2, E-cadherin, UTF-1, Oct4, Rex1, and Nanog. Cell morphologies associated with pluripotent stem cells are also pluripotent stem cell characteristics. As described herein, cells do not need to pass through pluripotency to be reprogrammed into endodermal progenitor cells and/or hepatocytes.

As used herein, “multipotent” or “multipotent cell” refers to a cell type that can give rise to a limited number of other particular cell types. For example, induced multipotent cells are capable of forming endodermal cells. Additionally, multipotent blood stem cells can differentiate itself into several types of blood cells, including lymphocytes, monocytes, neutrophils, etc.

As used herein, the term “oligopotent” refers to the ability of an adult stem cell to differentiate into only a few different cell types. For example, lymphoid or myeloid stem cells are capable of forming cells of either the lymphoid or myeloid lineages, respectively.

As used herein, the term “unipotent” means the ability of a cell to form a single cell type. For example, spermatogonial stem cells are only capable of forming sperm cells.

As used herein, the term “totipotent” means the ability of a cell to form an entire organism. For example, in mammals, only the zygote and the first cleavage stage blastomeres are totipotent.

As used herein, “non-pluripotent cells” refer to mammalian cells that are not pluripotent cells. Examples of such cells include differentiated cells as well as progenitor cells. Examples of differentiated cells include, but are not limited to, cells from a tissue selected from bone marrow, skin, skeletal muscle, fat tissue and peripheral blood. Exemplary cell types include, but are not limited to, fibroblasts, hepatocytes, myoblasts, neurons, osteoblasts, osteoclasts, and T-cells. The starting cells employed for generating the induced multipotent cells, the endodermal progenitor cells, and the hepatocytes can be non-pluripotent cells.

Differentiated cells include, but are not limited to, multipotent cells, oligopotent cells, unipotent cells, progenitor cells, and terminally differentiated cells. In particular embodiments, a less potent cell is considered “differentiated” in reference to a more potent cell.

A “somatic cell” is a cell forming the body of an organism. Somatic cells include cells making up organs, skin, blood, bones and connective tissue in an organism, but not germ cells.

Cells can be from, for example, human or non-human mammals. Exemplary non-human mammals include, but are not limited to, mice, rats, cats, dogs, rabbits, guinea pigs, hamsters, sheep, pigs, horses, bovines, and non-human primates. In some embodiments, a cell is from an adult human or non-human mammal. In some embodiments, a cell is from a neonatal human, an adult human, or non-human mammal.

As used herein, the terms “subject” or “patient” refers to any animal, such as a domesticated animal, a zoo animal, or a human. The “subject” or “patient” can be a mammal like a dog, cat, bird, livestock, or a human. Specific examples of “subjects” and “patients” include, but are not limited to, individuals (particularly human) with a disease or disorder related to the liver, heart, lung, kidney, pancreas, brain, neural tissue, blood, bone, bone marrow, and the like.

Mammalian cells can be from humans or non-human mammals. Exemplary non-human mammals include, but are not limited to, mice, rats, cats, dogs, rabbits, guinea pigs, hamsters, sheep, pigs, horses, bovines, and non-human primates (e.g., chimpanzees, macaques, and apes).

By “hypo-immunogenic pluripotent cell,” “hypoimmune pluripotent stem cell,” “hypoimmune pluripotent cell,” or “HIP cell” herein is meant a pluripotent cell that retains its pluripotent characteristics and yet gives rise to a reduced immunological rejection response when transferred into an allogeneic host. In preferred embodiments, HIP cells do not give rise to an immune response. Thus, “hypo-immunogenic” or “hypoimmune” refers to a significantly reduced or eliminated immune response when compared to the immune response of a parental (i.e. “wild-type” or “wt”) cell prior to immunoengineering as outlined herein. In many cases, the HIP cells are immunologically silent and yet retain pluripotent capabilities. Assays for HIP characteristics are outlined below.

By “HLA” or “human leukocyte antigen” complex is a gene complex encoding the major histocompatibility complex (MHC) proteins in humans. These cell-surface proteins that make up the HLA complex are responsible for the regulation of the immune response to antigens. In humans, there are two MHCs, class I and class II, “HLA-I” and “HLA-II”. HLA-I includes three proteins, HLA-A, HLA-B and HLA-C, which present peptides from the inside of the cell, and antigens presented by the HLA-I complex attract killer T-cells (also known as CD8+ T-cells or cytotoxic T cells). The HLA-I proteins are associated with β-2 microglobulin (B2M). HLA-II includes five proteins, HLA-DP, HLA-DM, HLA-DOB, HLA-DQ and HLA-DR, which present antigens from outside the cell to T lymphocytes. This stimulates CD4+ cells (also known as T-helper cells). It should be understood that the use of either “MHC” or “HLA” is not meant to be limiting, as it depends on whether the genes are from humans (HLA) or murine (MHC). Thus, as it relates to mammalian cells, these terms may be used interchangeably herein.

By “gene knock out” herein is meant a process that renders a particular gene inactive in the host cell in which it resides, resulting either in no protein of interest being produced or an inactive form. As will be appreciated by those in the art and further described below, this can be accomplished in a number of different ways, including removing nucleic acid sequences from a gene, or interrupting the sequence with other sequences, altering the reading frame, or altering the regulatory components of the nucleic acid. For example, all or part of a coding region of the gene of interest can be removed or replaced with “nonsense” sequences, all or part of a regulatory sequence such as a promoter can be removed or replaced, translation initiation sequences can be removed or replaced, etc.

By “gene knock in” herein is meant a process that adds a genetic function to a host cell. This causes increased levels of the encoded protein. As will be appreciated by those in the art, this can be accomplished in several ways, including adding one or more additional copies of the gene to the host cell or altering a regulatory component of the endogenous gene increasing expression of the protein is made. This may be accomplished by modifying the promoter, adding a different promoter, adding an enhancer, or modifying other gene expression sequences.

“β-2 microglobulin” or “β2M” or “B2M” protein refers to the human β2M protein that has the amino acid and nucleic acid sequences shown below; the human gene has accession number NC_000015.10:44711487-44718159.

“CD47 protein” protein refers to the human CD47 protein that has the amino acid and nucleic acid sequences shown below; the human gene has accession number NC_000003.12:108043094-108094200.

“CIITA protein” protein refers to the human CIITA protein that has the amino acid and nucleic acid sequences shown below; the human gene has accession number NC_000016.10:10866208-10941562.

By “wild type” in the context of a cell means a cell found in nature. However, in the context of a pluripotent stem cell, as used herein, it also means an iPSC that may contain nucleic acid changes resulting in pluripotency but did not undergo the gene editing procedures of the invention to achieve hypo-immunogenicity.

By “syngeneic” herein refers to the genetic similarity or identity of a host organism and a cellular transplant where there is immunological compatibility; e.g. no immune response is generated.

By “allogeneic” herein refers to the genetic dissimilarity of a host organism and a cellular transplant where an immune response is generated.

By “B2M−/−” herein is meant that a diploid cell has had the B2M gene inactivated in both chromosomes. As described herein, this can be done in a variety of ways.

By “CIITA−/−” herein is meant that a diploid cell has had the CIITA gene inactivated in both chromosomes. As described herein, this can be done in a variety of ways.

By “CD47 tg” (standing for “transgene”) or “CD47+”) herein is meant that the host cell expresses CD47, in some cases by having at least one additional copy of the CD47 gene.

An “Oct polypeptide” refers to any of the naturally-occurring members of Octamer family of transcription factors, or variants thereof that maintain transcription factor activity, similar (within at least 50%, 80%, or 90% activity) compared to the closest related naturally occurring family member, or polypeptides comprising at least the DNA-binding domain of the naturally occurring family member, and can further comprise a transcriptional activation domain. Exemplary Oct polypeptides include Oct-1, Oct-2, Oct-3/4, Oct-6, Oct-7, Oct-8, Oct-9, and Oct-11. Oct3/4 (referred to herein as “Oct4”) contains the POU domain, a 150 amino acid sequence conserved among Pit-1, Oct-1, Oct-2, and uric-86. (See, Ryan, A. K. & Rosenfeld, M. G., Genes Dev. 11:1207-1225 (1997), incorporated herein by reference in its entirety.) In some embodiments, variants have at least 85%, 90%, or 95% amino acid sequence identity across their whole sequence compared to a naturally occurring Oct polypeptide family member such as to those listed above or such as listed in GenBank accession number NP-002692.2 (human Oct4) or NP-038661.1 (mouse Oct4). Oct polypeptides (e.g., Oct3/4 or Oct 4) can be from human, mouse, rat, bovine, porcine, or other animals. Generally, the same species of protein will be used with the species of cells being manipulated. The Oct polypeptide(s) can be a pluripotency factor that can help induce multipotency in non-pluripotent cells.

A “Klf polypeptide” refers to any of the naturally-occurring members of the family of Krüppel-like factors (Klfs), zinc-finger proteins that contain amino acid sequences similar to those of the Drosophila embryonic pattern regulator Krüppel, or variants of the naturally-occurring members that maintain transcription factor activity similar (within at least 50%, 80%, or 90% activity) compared to the closest related naturally occurring family member, or polypeptides comprising at least the DNA-binding domain of the naturally occurring family member, and can further comprise a transcriptional activation domain. (See, Dang, D. T., Pevsner, J. & Yang, V. W., Cell Biol. 32:1103-1121 (2000), incorporated by reference herein in its entirety.) Exemplary Klf family members include, Klf1, Klf2, Klf3, Klf-4, Klf5, Klf6, Klf7, Klf8, Klf9, Klf10, Klf11, Klf12, Klf13, Klf14, Klf15, Klf16, and Klf17. Klf2 and Klf-4 were found to be factors capable of generating iPS cells in mice, and related genes Klf1 and Klf5 did as well, although with reduced efficiency. (See, Nakagawa, et al., Nature Biotechnology 26:101-106 (2007), incorporated by reference herein in its entirety.) In some embodiments, variants have at least 85%, 90%, or 95% amino acid sequence identity across their whole sequence compared to a naturally occurring Klf polypeptide family member such as to those listed above or such as listed in GenBank accession number CAX16088 (mouse Klf4) or CAX14962 (human Klf4). Klf polypeptides (e.g., Klf1, Klf4, and Klf5) can be from human, mouse, rat, bovine, porcine, or other animals. Generally, the same species of protein will be used with the species of cells being manipulated. The Klf polypeptide(s) can be a pluripotency factor. The expression of the Klf4 gene or polypeptide can help induce multipotency in a starting cell or a population of starting cells.

A “Myc polypeptide” refers to any of the naturally-occurring members of the Myc family. (See, e.g., Adhikary, S. & Eilers, M., Nat. Rev. Mol. Cell Biol. 6:635-645 (2005), incorporated by reference herein in its entirety.) It also includes variants that maintain similar transcription factor activity when compared to the closest related naturally occurring family member (i.e., within at least 50%, 80%, or 90% activity). It further includes polypeptides comprising at least the DNA-binding domain of a naturally occurring family member, and can further comprise a transcriptional activation domain. Exemplary Myc polypeptides include, e.g., c-Myc, N-Myc and L-Myc. In some embodiments, variants have at least 85%, 90%, or 95% amino acid sequence identity across their whole sequence compared to a naturally occurring Myc polypeptide family member, such as to those listed above or such as listed in Genbank accession number CAA25015 (human Myc). Myc polypeptides (e.g., c-Myc) can be from human, mouse, rat, bovine, porcine, or other animals. Generally, the same species of protein will be used with the species of cells being manipulated. The Myc polypeptide(s) can be a pluripotency factor.

A “Sox polypeptide” refers to any of the naturally-occurring members of the SRY-related HMG-box (Sox) transcription factors, characterized by the presence of the high-mobility group (HMG) domain, or variants thereof that maintain similar transcription factor activity when compared to the closest related naturally occurring family member (i.e. within at least 50%, 80%, or 90% activity). It also includes polypeptides comprising at least the DNA-binding domain of the naturally occurring family member, and can further comprise a transcriptional activation domain. (See, e.g., Dang, D. T. et al., Int. J. Biochem. Cell Biol. 32:1103-1121 (2000), incorporated by reference herein in its entirety.) Exemplary Sox polypeptides include, e.g., Sox1, Sox-2, Sox3, Sox4, Sox5, Sox6, Sox7, Sox8, Sox9, Sox10, Sox11, Sox12, Sox13, Sox14, Sox15, Sox17, Sox18, Sox-21, and Sox30. Sox1 has been shown to yield iPS cells with a similar efficiency as Sox2, and genes Sox3, Sox15, and Sox18 have also been shown to generate iPS cells, although with somewhat less efficiency than Sox2. (See, Nakagawa, et al., Nature Biotechnology 26:101-106 (2007), incorporated by reference herein in its entirety.) In some embodiments, variants have at least 85%, 90%, or 95% amino acid sequence identity across their whole sequence compared to a naturally occurring Sox polypeptide family member such as to those listed above or such as listed in Genbank accession number CAA83435 (human Sox2). Sox polypeptides (e.g., Sox1, Sox2, Sox3, Sox15, or Sox18) can be from human, mouse, rat, bovine, porcine, or other animals. Generally, the same species of protein will be used with the species of cells being manipulated. The Sox polypeptide(s) can be a pluripotency factor. As discussed herein, SOX2 proteins find particular use in the generation of iPSCs.

By “differentiated hypoimmunogenic pluripotent cells” or “differentiated HIP cells” or “dHIP cells” herein is meant iPS cells that have been engineered to possess hypoimmunogenicity (e.g., by the knock out of B2M and CIITA and the knock in of CD47) and then are differentiated into a cell type for ultimate transplantation into subjects. Thus, for example HIP cells can be differentiated into hepatocytes (“dHIP hepatocytes”), into beta-like pancreatic cells or islet organoids (“dHIP beta cells”), into endothelial cells (“dHIP endothelial cells”), etc.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

“Inhibitors,” “activators,” and “modulators” affect a function or expression of a biologically-relevant molecule. The term “modulator” includes both inhibitors and activators. They may be identified using in vitro and in vivo assays for expression or activity of a target molecule.

“Inhibitors” refer to agents that, e.g., inhibit expression or bind to target molecules or proteins. They may partially or totally block stimulation or have protease inhibitor activity. They may reduce, decrease, prevent, or delay activation, including inactivation, desensitizion, or down regulation of the activity of the described target protein. Modulators may be antagonists of the target molecule or protein.

“Activators” refer to agents that, e.g., induce or activate the function or expression of a target molecule or protein. They may bind to, stimulate, increase, open, activate, or facilitate the target molecule activity. Activators may be agonists of the target molecule or protein.

“Homologs” are bioactive molecules that are similar to a reference molecule at the nucleotide sequence, peptide sequence, functional, or structural level. Homologs may include sequence derivatives that share a certain percent identity with the reference sequence. Thus, in one embodiment, homologous or derivative sequences share at least a 70 percent sequence identity. In a specific embodiment, homologous or derivative sequences share at least an 80 or 85 percent sequence identity. In a specific embodiment, homologous or derivative sequences share at least a 90 percent sequence identity. In a specific embodiment, homologous or derivative sequences share at least a 95 percent sequence identity. In a more specific embodiment, homologous or derivative sequences share at least a 50, 55, 60, 65, 70, 75, 85, 86, 87, 88, 89, 90, 91, 92, 93, 94, 95, 96, 97, 98, or 99 percent sequence identity. Homologous or derivative nucleic acid sequences may also be defined by their ability to remain bound to a reference nucleic acid sequence under high stringency hybridization conditions. Homologs having a structural or functional similarity to a reference molecule may be chemical derivatives of the reference molecule. Methods of detecting, generating, and screening for structural and functional homologs as well as derivatives are known in the art.

“Hybridization” generally depends on the ability of denatured DNA to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al, Current Protocols in Molecular Biology, Wiley Interscience Publishers (1995), incorporated by reference herein in its entirety.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures.

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 Mm sodium phosphate buffer at pH 6.5 with 750 Mm sodium chloride, 75 Mm sodium citrate at 42° C.; or (3) overnight hybridization in a solution that employs 50% formamide, 5×SSC (0.75 M NaCl, 0.075 M sodium citrate), 50 Mm sodium phosphate (pH 6.8), 0.1% sodium pyrophosphate, 5×Denhardt's solution, sonicated salmon sperm DNA (50 μl/ml), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used herein the term “modification” refers to an alteration that physically differentiates the modified molecule from the parent molecule. In one embodiment, an amino acid change in a CD47, HSVtk, EC-CD, or iCasp9 variant polypeptide prepared according to the methods described herein differentiates it from the corresponding parent that has not been modified according to the methods described herein, such as wild-type proteins, a naturally occurring mutant proteins or another engineered protein that does not include the modifications of such variant polypeptide. In another embodiment, a variant polypeptide includes one or more modifications that differentiates the function of the variant polypeptide from the unmodified polypeptide. For example, an amino acid change in a variant polypeptide affects its receptor binding profile. In other embodiments, a variant polypeptide comprises substitution, deletion, or insertion modifications, or combinations thereof. In another embodiment, a variant polypeptide includes one or more modifications that increases its affinity for a receptor compared to the affinity of the unmodified polypeptide.

In one embodiment, a variant polypeptide includes one or more substitutions, insertions, or deletions relative to a corresponding native or parent sequence. In certain embodiments, a variant polypeptide includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31-40, 41 to 50, or 51 or more modifications.

By “episomal vector” herein is meant a genetic vector that can exist and replicate autonomously in the cytoplasm of a cell; e.g. it is not integrated into the genomic DNA of the host cell. A number of episomal vectors are known in the art and described below.

By “knock out” in the context of a gene means that the host cell harboring the knock out does not produce a functional protein product of the gene. As outlined herein, a knock out can result in a variety of ways, from removing all or part of the coding sequence, introducing frameshift mutations such that a functional protein is not produced (either truncated or nonsense sequence), removing or altering a regulatory component (e.g. a promoter) such that the gene is not transcribed, preventing translation through binding to mRNA, etc. Generally, the knock out is effected at the genomic DNA level, such that the cells' offspring also carry the knock out permanently.

By “knock in” in the context of a gene means that the host cell harboring the knock in has more functional protein active in the cell. As outlined herein, a knock in can be done in a variety of ways, usually by the introduction of at least one copy of a transgene (tg) encoding the protein into the cell, although this can also be done by replacing regulatory components as well, for example by adding a constitutive promoter to the endogeneous gene. In general, knock in technologies result in the integration of the extra copy of the transgene into the host cell.

### VII. Hypoimmunogenic Pluripotent (HIP) Cells

The invention provides compositions and methodologies for generating HIP cells, starting with wild type cells, rendering them pluripotent (e.g. making induced pluripotent stem cells, or iPSCs), then generating HIP cells from the iPSC population.

A. Methodologies for Genetic Alterations

The invention includes methods of modifying nucleic acid sequences within cells or in cell-free conditions to generate both pluripotent cells and HIP cells. Exemplary technologies include homologous recombination, knock-in, ZFNs (zinc finger nucleases), TALENs (transcription activator-like effector nucleases), meganucleases (e.g., homing endonucleases), CRISPR (clustered regularly interspaced short palindromic repeats)/Cas9, and other site-specific nuclease technologies. These techniques enable double-strand DNA breaks at desired locus sites. These controlled double-strand breaks promote homologous recombination at the specific locus sites. This process focuses on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR).

As will be appreciated by those in the art, a number of different techniques can be used to engineer the pluripotent cells of the invention, as well as the engineering of the iPSCs to become hypoimmunogenic as outlined herein.

In general, these techniques can be used individually or in combination. For example, in the generation of the HIP cells, CRISPR/Cas may be used to reduce the expression of active B2M and/or CIITA protein in the engineered cells, with viral techniques (e.g., retrovirus, lentivirus, and adeno-associated virus) to knock in the CD47 functionality. Also, as will be appreciated by those in the art, although one embodiment sequentially utilizes a CRISPR/Cas step to knock out B2M, followed by a CRISPR/Cas step to knock out CIITA with a final step of a lentivirus to knock in the CD47 functionality, these genes can be manipulated in different orders using different technologies.

As is discussed more fully below, transient expression of reprogramming genes is generally done to generate induced pluripotent stem cells.

a. CRISPR/Cas Technologies

In one embodiment, the cells are manipulated using clustered regularly interspaced short palindromic repeats)/Cas (“CRISPR”) technologies as is known in the art. CRISPR/Cas can be used to generate the starting iPSCs or to generate the HIP cells from the iPSCs. There are a large number of techniques based on CRISPR/Cas, see for example Doudna and Charpentier, Science doi:10.1126/science.1258096, hereby incorporated by reference. CRISPR techniques and kits are sold commercially.

b. TALEN Technologies

In some embodiments, the HIP cells of the invention are made using Transcription Activator-Like Effector Nucleases (TALEN) methodologies. TALEN are restriction enzymes combined with a nuclease that can be engineered to bind to and cut practically any desired DNA sequence. TALEN kits are sold commercially.

c. Zinc Finger Technologies

In one embodiment, the cells are manipulated using Zn finger nuclease technologies. Zn finger nucleases are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms, similar to CRISPR and TALENs.

d. Viral Based Technologies

There are a wide variety of viral techniques that can be used to generate the HIP cells of the invention (as well as for the original generation of the iPSCs), including, but not limited to, the use of retroviral vectors, lentiviral vectors, adenovirus vectors and Sendai viral vectors. Episomal vectors used in the generation of iPSCs are described below.

e. Downregulation of Genes Using Interfering RNA

In other embodiments, genes that encode proteins used in HLA molecules are downregulated by RNA interference (RNAi) technologies. RNAi refers to a process where RNA molecules inhibit gene expression often by causing specific mRNA molecules to degrade. Two types of RNA molecules—microRNA (miRNA) and small interfering RNA (siRNA)—can be used for RNA interference. They bind to the target mRNA molecules and either increase or decrease their activity. RNAi helps cells defend against parasitic nucleic acids such as those from viruses and transposons. RNAi also influences development.

According to particular embodiments, the inhibitory nucleic acid is an antisense oligonucleotide which inhibits the expression of a target gene, e.g., B2M gene and a CIITA gene. Such an antisense oligonucleotide can be a nucleic acid (either DNA or RNA) which specifically hybridizes (e.g., binds) under cellular conditions with the cellular mRNA and/or genomic DNA encoding the target protein, thereby inhibiting transcription and/or translation of the gene. The binding may be by conventional base pair complementarity. Alternatively, the binding may be, for example, in case of binding to DNA duplexes, through specific interactions in the major groove of the double helix. Absolute complementarity, although preferred, is not required.

Thus, according to an embodiment, the antisense oligonucleotide is a single-stranded or double-stranded DNA molecule, more preferably a double-stranded DNA molecule. According to another embodiment, the antisense oligonucleotide is a single-stranded or double-stranded RNA molecule, more preferably a single-stranded RNA molecule. In some instances, the antisense oligonucleotide is a modified oligonucleotide which is resistant to endogenous nucleases, e.g., exonucleases and/or endonucleases, and is therefore stable in vivo and in vitro.

The antisense oligonucleotide may be modified at the base moiety, sugar moiety, or phosphate backbone, for example, to improve stability of the molecule. The antisense oligonucleotide may include other appended groups such as peptides (e.g., for targeting host cell receptors), or agents facilitating transport across the cell membrane. The antisense oligonucleotide may be conjugated to another molecule such as a peptide or transport agent. In some cases, the antisense oligonucleotide comprises at least one modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxytriethyl) uracil, 5-carboxymethylaminomethyl-2-thiouridine, 5-carboxymethylanninonnethyluracil, dihydrouracil, beta-D-galactosylqueosine, inosine, N6-isopentenyladenine, 1-nnethylguanine, 1-methylinosine, 2,2-dimethylguanine, 2-methyladenine, 2-methylguanine, 3-methylcytosine, 5-methylcytosine, N6-adenine, 7-nnethylguanine, 5-methylaminonnethyluracil, 5-methoxyaminomethyl-2-thiouracil, beta-D-mannosylqueosine, 5-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyl uracil, uracil-5-oxyacetic acid methylester, uracil-5-oxyacetic acid (v), 5-methyl-2-thiouracil, 3-(3-amino-3-N-2-carboxypropyl) uracil, (acp3)w and 2,6-diaminopurine.

In certain embodiments, the antisense oligonucleotide comprise at least one modified sugar moiety selected from the group including, but not limited to, arabinose, 2-fluoroara binose, xylulose and hexose. In other embodiments, the antisense oligonucleotide comprises at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof.

sdRNA molecules are a class of asymmetric siRNAs comprising a guide (antisense) strand of 19-21 bases. They can contain a 5′ phosphate, 2′Ome or 2′F modified pyrimidines, and six phosphorotioates at the 3′ positions. They can contain a sense strand containing 3′ conjugated sterol moieties, 2 phosphotioates at the 3′ position, and 2′Ome modified pyrimidines. Both strands can contain 2′ Ome purines with continuous stretches of unmodified purines not exceeding a length of 3. sdRNA is disclosed in U.S. Pat. No. 8,796,443, incorporated herein by reference in its entirety.

For all of these technologies, well known recombinant techniques are used, to generate recombinant nucleic acids as outlined herein. In certain embodiments, the recombinant nucleic acids (either than encode a desired polypeptide, e.g. CD47, or disruption sequences) may be operably linked to one or more regulatory nucleotide sequences in an expression construct. Regulatory nucleotide sequences will generally be appropriate for the host cell and subject to be treated. Numerous types of appropriate expression vectors and suitable regulatory sequences are known in the art for a variety of host cells. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, or hybrid promoters that combine elements of more than one promoter. An expression construct may be present in a cell on an episome, such as a plasmid or vector, or the expression construct may be inserted in a chromosome. In a specific embodiment, the expression vector includes a selectable marker gene to allow the selection of transformed host cells. Certain embodiments include an expression vector comprising a nucleotide sequence encoding a variant polypeptide operably linked to at least one regulatory sequence. Regulatory sequence for use herein include promoters, enhancers, and other expression control elements. In certain embodiments, an expression vector is designed for the choice of the host cell to be transformed, the particular variant polypeptide desired to be expressed, the vector's copy number, the ability to control that copy number, or the expression of any other protein encoded by the vector, such as antibiotic markers.

Examples of suitable promoters include, for example, promoters from the following genes: ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s). In some embodiments, the elongation factor 1-alpha promoter is used.

In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication. Fiers et al., Nature 273: 113-120 (1978). The immediate early promoter of the human cytomegalovirus is conveniently obtained as a HindIII E restriction fragment. Greenaway, P. J. et al., Gene 18: 355-360 (1982). The foregoing references are incorporated by reference in their entirety.

B. Generation of Pluripotent Cells

The invention provides methods of producing non-immunogenic pluripotent cells from pluripotent cells. Thus, the first step is to provide the pluripotent stem cells.

The generation of mouse and human pluripotent stem cells (generally referred to as iPSCs; miPSCs for murine cells or hiPSCs for human cells) is generally known in the art. As will be appreciated by those in the art, there are a variety of different methods for the generation of iPSCs. The original induction was done from mouse embryonic or adult fibroblasts using the viral introduction of four transcription factors, Oct3/4, Sox2, c-Myc and Klf4; see Takahashi and Yamanaka Cell 126:663-676 (2006), hereby incorporated by reference in its entirety and specifically for the techniques outlined therein. Since then, a number of methods have been developed; see Seki et al., World J. Stem Cells 7(1):116-125 (2015) for a review, and Lakshmipathy and Vermuri, editors, Methods in Molecular Biology: Pluripotent Stem Cells, Methods and Protocols, Springer 2013, both of which are hereby expressly incorporated by reference in their entirety, and in particular for the methods for generating hiPSCs (see for example Chapter 3 of the latter reference).

Generally, iPSCs are generated by the transient expression of one or more “reprogramming factors” in the host cell, usually introduced using episomal vectors. Under these conditions, small amounts of the cells are induced to become iPSCs (in general, the efficiency of this step is low, as no selection markers are used). Once the cells are “reprogrammed”, and become pluripotent, they lose the episomal vector(s) and produce the factors using the endogeneous genes. This loss of the episomal vector(s) results in cells that are called “zero footprint” cells. This is desirable as the fewer genetic modifications (particularly in the genome of the host cell), the better. Thus, it is preferred that the resulting hiPSCs have no permanent genetic modifications.

As is also appreciated by those of skill in the art, the number of reprogramming factors that can be used or are used can vary. Commonly, when fewer reprogramming factors are used, the efficiency of the transformation of the cells to a pluripotent state goes down, as well as the “pluripotency”, e.g. fewer reprogramming factors may result in cells that are not fully pluripotent but may only be able to differentiate into fewer cell types.

In some embodiments, a single reprogramming factor, OCT4, is used. In other embodiments, two reprogramming factors, OCT4 and KLF4, are used. In other embodiments, three reprogramming factors, OCT4, KLF4 and SOX2, are used. In other embodiments, four reprogramming factors, OCT4, KLF4, SOX2 and c-Myc, are used. In other embodiments, 5, 6 or 7 reprogramming factors can be used selected from SOKMNLT: SOX2, OCT4 (POU5F1), KLF4, MYC, NANOG, LIN28, and SV40L T antigen.

In general, these reprogramming factor genes are provided on episomal vectors such as are known in the art and commercially available. For example, ThermoFisher/Invitrogen sell a sendai virus reprogramming kit for zero footprint generation of hiPSCs, see catalog number A34546. ThermoFisher also sells EBNA-based systems as well, see catalog number A14703.

In addition, there are a number of commercially available hiPSC lines available; see, e.g., the Gibco® Episomal hiPSC line, K18945, which is a zero footprint, viral-integration-free human iPSC cell line (see also Burridge et al, 2011, supra).

In general, as is known in the art, iPSCs are made from non-pluripotent cells such as CD34+ cord blood cells, fibroblasts, etc., by transiently expressing the reprogramming factors as described herein.

For example, successful iPSCs were also generated using only Oct3/4, Sox2 and Klf4, while omitting the C-Myc, although with reduced reprogramming efficiency.

In general, iPSCs are characterized by the expression of certain factors that include KLF4, Nanog, OCT4, SOX2, ESRRB, TBX3, c-Myc and TCL1. New or increased expression of these factors for purposes of the invention may be via induction or modulation of an endogenous locus or from expression from a transgene.

For example, murine iPSCs can be generated using the methods of Diecke et al, Sci Rep. 2015, Jan. 28; 5:8081 (doi:10.1038/srep08081), hereby incorporated by reference in its entirety and specifically for the methods and reagents for the generation of the miPSCs. See also, e.g., Burridge et al., PLoS One, 2011 6(4):18293, hereby incorporated by reference in its entirety and specifically for the methods outlined therein.

In some cases, the pluripotency of the cells is measured or confirmed as outlined herein, for example by assaying for reprogramming factors as is generally shown in PCT/US18/13688 or by conducting differentiation reactions as outlined therein, for instance, in the Examples.

### C. Generation of Hypo-Immunogenic Pluripotent (HIP) Cells

The present invention is directed to the generation, manipulation, growth and transplantation of hypo-immunogenic cells into a patient as defined herein. The generation of HIP cells from pluripotent cells is done with as few as three genetic changes, resulting in minimal disruption of cellular activity but conferring immunosilencing to the cells.

As discussed herein, one embodiment utilizes a reduction or elimination in the protein activity of MHC I and II (HLA I and II when the cells are human). This can be done by altering genes encoding their component. In one embodiment, the coding region or regulatory sequences of the gene are disrupted using CRISPR/Cas. In another embodiment, gene translation is reduced using interfering RNA technologies. The third change is a change in a gene that regulates susceptibility to macrophage phagocytosis, such as CD47, and this is generally a “knock in” of a gene using viral technologies.

Additional descriptions of HIP cells can be found in International Application No. PCT/US18/13688, filed on Jan. 14, 2018 and U.S. Provisional Application No. 62/445,969, filed Jan. 13, 2017, the disclosures in their entirety are herein incorporated by reference, in particular, the examples, figures, figure descriptions, and descriptions of producing hypoimmunogenic pluripotent stem cells and differentiating such cells into other cell types.

In some cases, where CRISPR/Cas is being used for the genetic modifications, hiPSC cells that contain a Cas9 construct that enable high efficiency editing of the cell line can be used; see, e.g., the Human Episomal Cas9 iPSC cell line, A33124, from Life Technologies.

1. HLA-I Reduction

The HIP cells of the invention include a reduction in MHC I function (HLA I when the cells are derived from human cells).

As will be appreciated by those in the art, the reduction in function can be accomplished in a number of ways, including removing nucleic acid sequences from a gene, interrupting the sequence with other sequences, or altering the regulatory components of the nucleic acid. For example, all or part of a coding region of the gene of interest can be removed or replaced with “nonsense” sequences, frameshift mutations can be made, all or part of a regulatory sequence such as a promoter can be removed or replaced, translation initiation sequences can be removed or replaced, etc.

As will be appreciated by those in the art, the successful reduction of the MHC I function (HLA I when the cells are derived from human cells) in the pluripotent cells can be measured using techniques known in the art and as described below; for example, FACS techniques using labeled antibodies that bind the HLA complex; for example, using commercially available HLA-A, B, C antibodies that bind to the alpha chain of the human major histocompatibility HLA Class I antigens.

B2M Alteration

In one embodiment, the reduction in HLA-I activity is done by disrupting the expression of the β-2 microglobulin gene in the pluripotent stem cell, the human sequence of which is disclosed herein. This alteration is generally referred to herein as a gene “knock out”, and in the HIP cells of the invention it is done on both alleles in the host cell. Generally the techniques to do both disruptions is the same.

A particularly useful embodiment uses CRISPR technology to disrupt the gene. In some cases, CRISPR technology is used to introduce small deletions/insertions into the coding region of the gene, such that no functional protein is produced, often the result of frameshift mutations that result in the generation of stop codons such that truncated, non-functional proteins are made.

Accordingly, a useful technique is to use CRISPR sequences designed to target the coding sequence of the B2M gene in mouse or the B2M gene in human. After gene editing, the transfected iPSC cultures are dissociated to single cells. Single cells are expanded to full-size colonies and assessed for CRISPR/Cas edit by screening for presence of aberrant sequence from the CRISPR cleavage site. Clones with deletions in both alleles are picked. Such clones did not express B2M as demonstrated by PCR and did not express HLA-I as demonstrated by FACS analysis (see examples 1 and 6, for example of PCT/US18/13688).

Assays to test whether the B2M gene has been inactivated are known and described herein. In one embodiment, the assay is a Western blot oaf cells lysates probed with antibodies to the B2M protein. In another embodiment, reverse transcriptase polymerase chain reactions (RT-PCR) confirms the presence of the inactivating alteration.

In addition, the cells can be assessed to confirm that the HLA I complex is not expressed on the cell surface. This may be assayed by FACS analysis using antibodies to one or more HLA cell surface components as discussed above.

It is noteworthy that others have had poor results when trying to silence the B2M genes at both alleles. See, e.g. Gornalusse et al., Nature Biotech. Doi/10.1038/nbt.3860).

2. HLA-II Reduction

In addition to a reduction in HLA I, the HIP cells of the invention also lack MHC II function (HLA II when the cells are derived from human cells).

As will be appreciated by those in the art, the reduction in function can be accomplished in a number of ways, including removing nucleic acid sequences from a gene, adding nucleic acid sequences to a gene, disrupting the reading frame, interrupting the sequence with other sequences, or altering the regulatory components of the nucleic acid. In one embodiment, all or part of a coding region of the gene of interest can be removed or replaced with “nonsense” sequences. In another embodiment, regulatory sequences such as a promoter can be removed or replaced, translation initiation sequences can be removed or replaced, etc.

The successful reduction of the MHC II function (HLA II when the cells are derived from human cells) in the pluripotent cells or their derivatives can be measured using techniques known in the art such as Western blotting using antibodies to the protein, FACS techniques, rt-PCR techniques, etc.

CIITA Alteration

In one embodiment, the reduction in HLA-II activity is done by disrupting the expression of the CIITA gene in the pluripotent stem cell, the human sequence of which is shown herein. This alteration is generally referred to herein as a gene “knock out”, and in the HIP cells of the invention it is done on both alleles in the host cell.

Assays to test whether the CIITA gene has been inactivated are known and described herein. In one embodiment, the assay is a Western blot of cells lysates probed with antibodies to the CIITA protein. In another embodiment, reverse transcriptase polymerase chain reactions RT-PCR) confirms the presence of the inactivating alteration.

In addition, the cells can be assessed to confirm that the HLA II complex is not expressed on the cell surface. Again, this assay is done as is known in the art (See FIG. 21 of PCT/US18/13688, for example) and generally is done using either Western Blots or FACS analysis based on commercial antibodies that bind to human HLA Class II HLA-DR, DP and most DQ antigens as outlined below.

A particularly useful embodiment uses CRISPR technology to disrupt the CIITA gene. CRISPRs were designed to target the coding sequence of the CIITA gene in mouse or the CIITA gene in human, an essential transcription factor for all MHC II molecules. After gene editing, the transfected iPSC cultures were dissociated into single cells. They were expanded to full-size colonies and assessed for successful CRISPR editing by screening for the presence of an aberrant sequence from the CRISPR cleavage site. Clones with deletions did not express CIITA as determined by PCR and did not express MHC II/HLA-II as determined by FACS analysis.

3. Reduction of Macrophage Phagocytosis and/or NK Cell Killing

In addition to the reduction of HLA I and II (or MHC I and II), generally using B2M and CIITA knock-outs, the HIP cells of the invention have a reduced susceptibility to macrophage phagocytosis and NK cell killing. The resulting HIP cells “escape” the immune macrophage and innate pathways due to the expression of one or more CD47 transgenes.

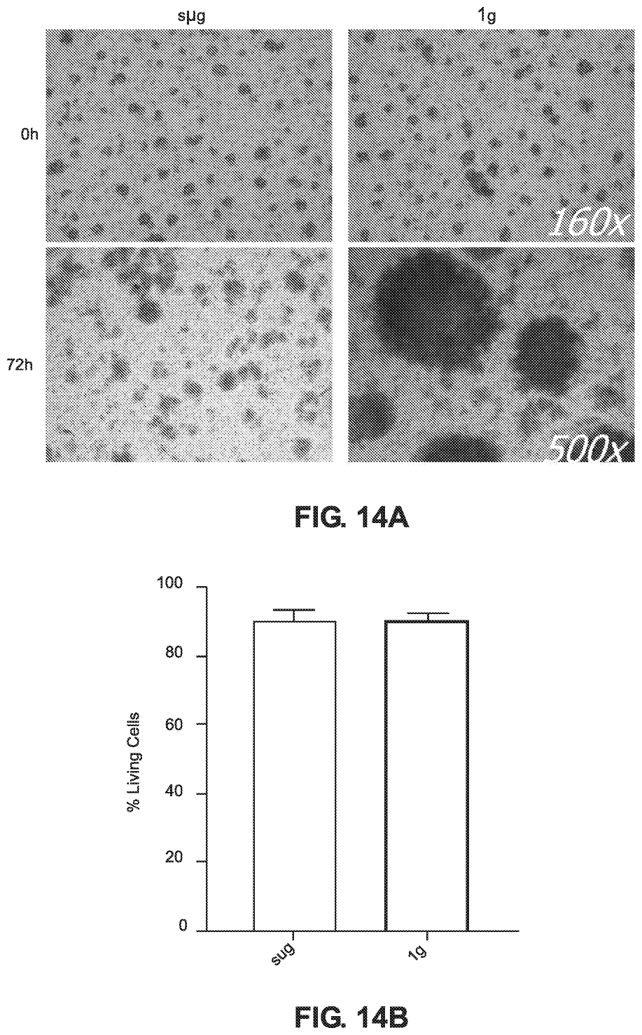

The ability of HIP cells and cells derived from the HIP cells to evade or escape NK cell killing and/or macrophage phagocytosis is shown in FIGS. 14A-14C and 34A-34C of PCT/US18/13688, the contents, in particular, the figures, figure descriptions, and examples are herein incorporated by reference. For example, FIGS. 14B-14C show that mouse HIP cells (e.g., B2m−/−Ciita−/−CD47 transgenic mouse iPSCs) failed to induce CD107a expression by NK cells, and thus did not elicit an NK cell response. In addition, it was shown that such mouse HIP cells did not induce activation of NK cells or release of IFNγ. When NK cells were incubated with differentiated cells (such as endothelial cells, smooth muscle cells, and cardiomyocytes) derived from HIP cells, NK cell responses were not induced (see, e.g., FIGS. 34A-34C of PCT/US18/13688).

Increased CD47 Expression

In some embodiments, reduced macrophage phagocytosis and NK cell killing susceptibility results from increased CD47 on the HIP cell surface. This is done in several ways as will be appreciated by those in the art using “knock in” or transgenic technologies. In some cases, increased CD47 expression results from one or more CD47 transgene.

Accordingly, in some embodiments, one or more copies of a CD47 gene is added to the HIP cells under control of an inducible or constitutive promoter, with the latter being preferred. In some embodiments, a lentiviral construct is employed as described herein or known in the art. CD47 genes may integrate into the genome of the host cell under the control of a suitable promoter as is known in the art.

The HIP cell lines were generated from B2M−/− CIITA−/− iPSCs. Cells containing lentivirus vectors expressing CD47 were selected using a Blasticidin marker. The CD47 gene sequence was synthesized and the DNA was cloned into the plasmid Lentivirus pLenti6N5 with a blasticidin resistance (Thermo Fisher Scientific, Waltham, Mass.)

In some embodiments, the expression of the CD47 gene can be increased by altering the regulatory sequences of the endogenous CD47 gene, for example, by exchanging the endogenous promoter for a constitutive promoter or for a different inducible promoter. This can generally be done using known techniques such as CRISPR.

Once altered, the presence of sufficient CD47 expression can be assayed using known techniques such as those described in the Examples, such as Western blots, ELISA assays or FACS assays using anti-CD47 antibodies. In general, “sufficiency” in this context means an increase in the expression of CD47 on the HIP cell surface that silences NK cell killing and/or macrophage phagocytosis. The natural expression levels on cells is too low to protect them from NK cell lysis once their MHC I is removed.

4. Suicide Genes

In some embodiments, the invention provides hypoimmunogenic pluripotent cells that comprise a “suicide gene” or “suicide switch”. These are incorporated to function as a “safety switch” that can cause the death of the hypoimmunogenic pluripotent cells should they grow and divide in an undesired manner. The “suicide gene” ablation approach includes a suicide gene in a gene transfer vector encoding a protein that results in cell killing only when activated by a specific compound. A suicide gene may encode an enzyme that selectively converts a nontoxic compound into highly toxic metabolites. The result is specifically eliminating cells expressing the enzyme. In some embodiments, the suicide gene is the herpesvirus thymidine kinase (HSV-tk) gene and the trigger is ganciclovir. In other embodiments, the suicide gene is the Escherichia coli cytosine deaminase (EC-CD) gene and the trigger is 5-fluorocytosine (5-FC) (Barese et al., Mol. Therap. 20(10):1932-1943 (2012), Xu et al., Cell Res. 8:73-8 (1998), both incorporated herein by reference in their entirety.)

In other embodiments, the suicide gene is an inducible Caspase protein. An inducible Caspase protein comprises at least a portion of a Caspase protein capable of inducing apoptosis. In one embodiment, the portion of the Caspase protein is exemplified in SEQ ID NO:6. In preferred embodiments, the inducible Caspase protein is iCasp9. It comprises the sequence of the human FK506-binding protein, FKBP12, with an F36V mutation, connected through a series of amino acids to the gene encoding human caspase 9. FKBP12-F36V binds with high affinity to a small-molecule dimerizing agent, AP1903. Thus, the suicide function of iCasp9 in the instant invention is triggered by the administration of a chemical inducer of dimerization (CID). In some embodiments, the CID is the small molecule drug AP1903. Dimerization causes the rapid induction of apoptosis. (See WO2011146862; Stasi et al, N Engl. J. Med 365; 18 (2011); Tey et al., Biol. Blood Marrow Transplant. 13:913-924 (2007), each of which are incorporated by reference herein in their entirety.)

5. Assays for HIP Phenotypes and Retention of Pluripotency

Once the HIP cells have been generated, they may be assayed for their hypo-immunogenicity and/or retention of pluripotency as is generally described herein and in the examples.

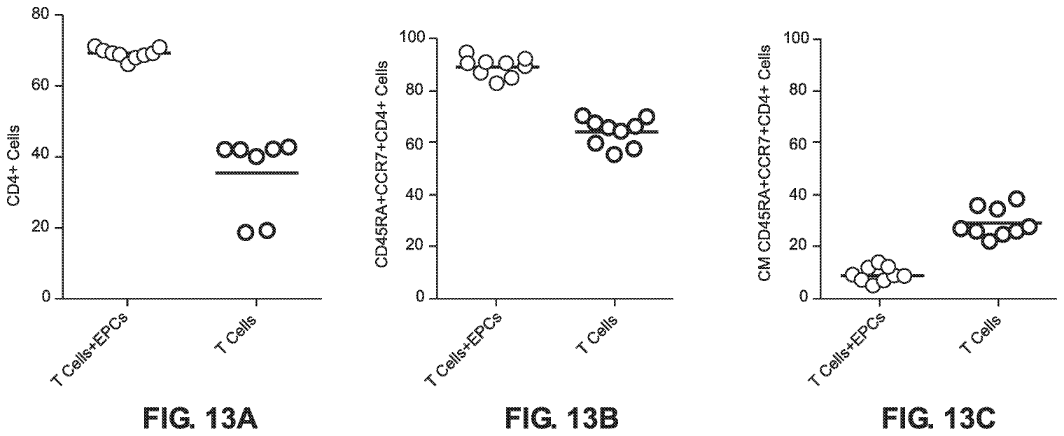

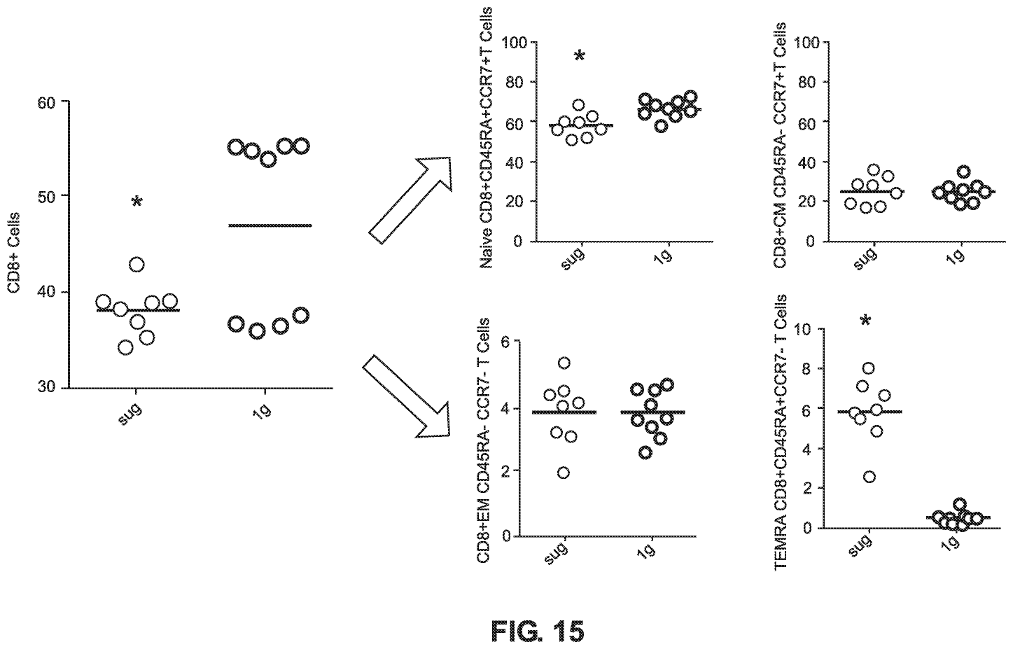

For example, hypo-immunogenicity are assayed using a number of techniques as exemplified in FIG. 13 and FIG. 15 of PCT/US18/13688. These techniques include transplantation into allogeneic hosts and monitoring for HIP cell growth (e.g. teratomas) that escape the host immune system. HIP derivatives are transduced to express luciferase and can then followed using bioluminescence imaging. Similarly, the T cell and/or B cell response of the host animal to the HIP cells are analyzed to confirm that the HIP cells do not cause an immune reaction in the host animal. T cell function is assessed by Elispot, ELISA, FACS, PCR, or mass cytometry (CYTOF). B cell response or antibody response is assessed using FACS or luminex. Additionally or alternatively, the cells may be assayed for their ability to avoid innate immune responses, e.g. NK cell killing, as is generally shown in FIGS. 14A-14C of PCT/US18/13688. NK cell lytolytic activity is assessed in vitro or in vivo (as shown in FIGS. 15A-15B of PCT/US18/13688).

Similarly, the retention of pluripotency is assessed in a number of ways. In one embodiment, pluripotency is assayed by the expression of certain pluripotency-specific factors as generally described herein and shown in FIG. 29 of PCT/US18/13688. In addition or alternatively, the HIP cells are differentiated into one or more cell types as an indication of pluripotency.

### D. Preferred Embodiments of the HIP Cells

Provided herein are hypoimmunogenic pluripotent stem cells (“HIP cells”) that exhibit pluripotency but do not result in a host immune response when transplanted into an allogeneic host such as a human patient, either as the HIP cells or as the differentiated products of the HIP cells.

In one embodiment, human pluripotent stem cells such as human induced pluripotent stem cells are rendered hypo-immunogenic by a) the disruption of the B2M gene at each allele (e.g., B2M−/−), b) the disruption of the CIITA gene at each allele (e.g. CIITA−/−), and c) by the overexpression of the CD47 gene (CD47+, e.g. through introducing one or more additional copies of the CD47 gene or activating the genomic gene). This renders the hiPSC population B2M−/− CIITA−/− CD47tg. In a preferred embodiment, the cells are non-immunogenic. In another embodiment, the HIP cells are rendered non-immunogenic B2M−/− CIITA−/− CD47 transgene as described above but are further modified by including an inducible suicide gene that is induced to kill the cells in vivo when required.

### E. Maintenance of HIP Cells

Once generated, the HIP cells can be maintained an undifferentiated state as is known for maintaining iPSCs. For example, HIP cells are cultured on Matrigel using culture media that prevents differentiation and maintains pluripotency.

### F. Differentiation of HIP Cells

HIP cells described herein can be differentiated into different cell types. The pluripotency of the HIPs can be evaluated by differentiating the cells into endodermal, mesodermal and ectodermal cell types. In some cases, the HIP cells are assessed by teratoma formation.

As will be appreciated by those in the art, the methods for differentiation depend on the desired cell type using known techniques. The cells are differentiated in suspension and then put into a gel matrix form, such as matrigel, gelatin, or fibrin/thrombin forms to facilitate cell survival. Differentiation is assayed as is known in the art, generally by evaluating the presence of cell-specific markers.

In some embodiments, the HIP cells are differentiated into hepatocytes to address loss of the hepatocyte functioning or cirrhosis of the liver. There are a number of techniques that can be used to differentiate HIP cells into hepatocytes; see for example Pettinato et al., doi:10.1038/spre32888, Snykers et al., Methods Mol Biol 698:305-314 (2011), Si-Tayeb et al, Hepatology 51:297-305 (2010) and Asgari et al., Stem Cell Rev (:493-504 (2013), all of which are hereby expressly incorporated by reference in their entirety and specifically for the methodologies and reagents for differentiation. Differentiation is assayed as is known in the art, generally by evaluating the presence of hepatocyte associated and/or specific markers, including, but not limited to, albumin, alpha fetoprotein, and fibrinogen. Differentiation can also be measured functionally, such as the metabolization of ammonia, LDL storage and uptake, ICG uptake and release and glycogen storage.

In some embodiments, the HIP cells are differentiated into beta-like cells or islet organoids for transplantation to address type I diabetes mellitus (T1DM). Cell systems are a promising way to address T1DM, see, e.g., Ellis et al., doi/10.1038/nrgastro.2017.93, incorporated herein by reference. Additionally, Pagliuca et al. reports on the successful differentiation of β-cells from hiPSCs (see doi/10.106/j.cell.2014.09.040, hereby incorporated by reference in its entirety and in particular for the methods and reagents outlined there for the large-scale production of functional human β cells from human pluripotent stem cells). Furthermore, Vegas et al. shows the production of human β cells from human pluripotent stem cells followed by encapsulation to avoid immune rejection by the host; (doi:10.1038/nm.4030, hereby incorporated by reference in its entirety and in particular for the methods and reagents outlined there for the large-scale production of functional human β cells from human pluripotent stem cells).

Differentiation is assayed as is known in the art, generally by evaluating the presence of β cell associated or specific markers, including but not limited to, insulin. Differentiation can also be measured functionally, such as measuring glucose metabolism, see generally Muraro et al, doi:10.1016/j.cels.2016.09.002, hereby incorporated by reference in its entirety, and specifically for the biomarkers outlined there.

Once the beta cells derived from HIP cells are generated, they can be transplanted (either as a cell suspension or within a gel matrix as discussed herein) into the portal vein/liver, the omentum, the gastrointestinal mucosa, the bone marrow, a muscle, or subcutaneous pouches.

In some embodiments, the HIP cells are differentiated into retinal pigment epithelium (RPE) to address sight-threatening diseases of the eye. Human pluripotent stem cells have been differentiated into RPE cells using the techniques outlined in Kamao et al., Stem Cell Reports 2014:2:205-18, hereby incorporated by reference in its entirety and in particular for the methods and reagents outlined there for the differentiation techniques and reagents; see also Mandai et al., doi:10.1056/NEJMoa1608368, also incorporated in its entirety for techniques for generating sheets of RPE cells and transplantation into patients.

Differentiation can be assayed as is known in the art, generally by evaluating the presence of RPE associated and/or specific markers or by measuring functionally. See for example Kamao et al., doi:10.1016/j.stemcr.2013.12.007, hereby incorporated by reference in its entirety and specifically for the markers outlined in the first paragraph of the results section.

In some embodiments, the HIP cells are differentiated into cardiomyocytes to address cardiovascular diseases. Techniques are known in the art for the differentiation of hiPSCs to cardiomyoctes and discussed in the Examples. Differentiation can be assayed as is known in the art, generally by evaluating the presence of cardiomyocyte associated or specific markers or by measuring functionally; see for example Loh et al., doi:10.1016/j.cell.2016.06.001, hereby incorporated by reference in its entirety and specifically for the methods of differentiating stem cells including cardiomyocytes.

In some embodiments, the HIP cells are differentiated into endothelial colony forming cells (ECFCs) to form new blood vessels to address peripheral arterial disease. Techniques to differentiate endothelial cells are known. See, e.g., Prasain et al., doi:10.1038/nbt.3048, incorporated by reference in its entirety and specifically for the methods and reagents for the generation of endothelial cells from human pluripotent stem cells, and also for transplantation techniques. Differentiation can be assayed as is known in the art, generally by evaluating the presence of endothelial cell associated or specific markers or by measuring functionally.

In some embodiments, the HIP cells are differentiated into thyroid progenitor cells and thyroid follicular organoids that can secrete thyroid hormones to address autoimmune thyroiditis. Techniques to differentiate thyroid cells are known the art. See, e.g. Kurmann et al., doi:10.106/j.stem.2015.09.004, hereby expressly incorporated by reference in its entirety and specifically for the methods and reagents for the generation of thyroid cells from human pluripotent stem cells, and also for transplantation techniques. Differentiation can be assayed as is known in the art, generally by evaluating the presence of thyroid cell associated or specific markers or by measuring functionally.

### VIII. HYPOIMMUNE CHIMERIC ANTIGEN RECEPTOR T CELLS DERIVED FROM HIP CELLS

The present invention provides an engineered T cell differentiated from a HIP cell comprising a nucleic acid encoding a CAR comprising an antigen binding domain, a transmembrane domain, and an intracellular signaling domain. In some embodiments, the CAR comprises an antigen binding domain, a transmembrane domain, and an intracellular signaling domain of a costimulatory domain. In another aspect of the invention, provided herein are hypoimmunogenic pluripotent cells comprising a nucleic acid encoding a CAR comprising an antigen binding domain, a transmembrane domain, and an intracellular signaling domain. Such hypoimmunogenic pluripotent cells can be in vitro differentiated to T cells to produce hypoimmunogenic CAR-T (HI-CAR-T) cells.

In some embodiments, the hypoimmunogenic CAR-T cells lack MHC I function or HLA-I function. For instance, the hypoimmunogenic CAR-T cells have reduced expression or lack expression of HLA-A protein, HLA-B protein, and HLA-C protein. In particular cases, the hypoimmunogenic CAR-T cells possess genetic modifications to inactivate the gene encoding HLA-A protein, the gene encoding HLA-B protein, the gene encoding HLA-C protein. In certain cases, the hypoimmunogenic CAR-T cells have reduced or lack expression of β-2 microglobulin protein. In some embodiments, such cells possess a genetic modification that inactivates the gene encoding β-2 microglobulin. Such hypoimmunogenic CAR-T cells can be differentiated from hypoimmunogenic pluripotent cells lack HLA-I function. In some embodiments, the hypoimmunogenic pluripotent cells possess a genetic modification that inactivates the gene encoding β-2 microglobulin.

In certain embodiments, the hypoimmunogenic CAR-T cells lack MHC II function or HLA-II function. In some instances, the hypoimmunogenic CAR-T cells have reduced expression or lack expression of HLA-DP protein, HLA-DR protein, and HLA-DQ protein. The hypoimmunogenic CAR-T cells may possess genetic modifications to inactivate the gene encoding HLA-DP protein, the gene encoding HLA-DR protein, the gene encoding HLA-DQ protein. In some embodiments, the hypoimmunogenic CAR-T cells have reduced or lack expression of CIITA protein. In some embodiments, such cells possess a genetic modification that inactivates the gene encoding CIITA. Such hypoimmunogenic CAR-T cells can be differentiated from hypoimmunogenic pluripotent cells lack HLA-II function. In some embodiments, the hypoimmunogenic pluripotent cells possess a genetic modification that inactivates the gene encoding CIITA.

In some embodiments, the hypoimmunogenic CAR-T cells have an increased expression of CD47 protein compared to a wild-type or native T cell. In other embodiments, the hypoimmunogenic pluripotent cells have an increased expression of CD47 protein compared to a wild-type or native pluripotent cell. Increased expression of CD47 may result from a genetic modification to an endogenous CD47 gene. In other cases, increased expression results from expression of an exogenous CD47 gene, e.g., an exogenous nucleic acid encoding CD47. Such hypoimmunogenic CAR-T cells can be differentiated from hypoimmunogenic pluripotent cells overexpressing CD47 protein. In some embodiments, the hypoimmunogenic pluripotent cells have increased expression of CD47 protein.

In some embodiments, the hypoimmunogenic CAR-T cell comprises a suicide gene such as, but not limited to, a herpes simplex virus thymidine kinase (HSV-tk) gene, an Escherichia coli cytosine deaminase (CD)gene, and a gene encoding an inducible caspase-9 protein. A suicide gene can be activated upon exposing the cell comprising the gene to a chemical agent (e.g., chemical trigger) that causes the cell to die. A chemical trigger for HSV-tk can be a dideoxynucleoside analog, e.g., ganciclovir. A chemical trigger for EC-CD can be 5-fluorocytosine (5-FC). A chemical trigger for caspase-9 can be a chemical inducer of dimerization (CID) such as the compound AP1903. Thus, the hypoimmunogenic pluripotent cell comprises the suicide gene and is differentiated to any one of the hypoimmunogenic CAR-T cells described herein.

Descriptions about a cytosine deaminase suicide gene system can be found, e.g., Mullin et al., Cancer Research, 1994, 54: 1503-1506. Details about a thymidine kinase suicide gene system can be found, e.g., Moolten, Cancer Research, 1986, 46(10): 5276-5281. Detailed descriptions of an inducible caspase-9 suicide gene system can be found, e.g., in Gargett and Brown, Front Pharmacol, 2014, 5:235.

A. Chimeric Antigen Receptors

In various embodiments, the antigen binding domain binds to an antigen on a target cell, e.g., a cancer cell. The antigen binding domain (also referred to as an extracellular domain) can bind antigens as is know in the art. In some embodiments, the antigen binding domain comprises a monoclonal antibody, a polyclonal antibody, a synthetic antibody, a human antibody, a humanized antibody, a non-human antibody, a nanobody, a single-chain variable fragment (scFv), F(ab′)2, Fab′, Fab, Fv, and the like.

The antigen binding domain can include a signal peptide. In addition, the CAR can contain a spacer region between the antigen binding domain the transmembrane domain. The spacer region should be flexible enough to allow the antigen binding domain to orient in different directions to facilitate antigen recognition. The spacer can be the hinge region from IgG1, or the CH2 and CH3 region of immunoglobulin and portions of CD3.

The antigen binding domain can be linked to the transmembrane domain of the CAR. In some embodiments, a nucleic acid encoding the antigen binding domain is operably linked to a nucleic acid encoding a transmembrane domain of the CAR.

In some embodiments, the transmembrane domain can be derived from a membrane-bound or transmembrane protein. In certain embodiments, the transmembrane domain comprises one or more, e.g., 1, 2, 3, 4, 5, 6, 7, 8 or more amino acid modifications (e.g., substitutions, insertions, and deletions) compared to the wild-type amino acid sequence of the transmembrane domain of the membrane-bound or transmembrane protein. Non-limiting examples of a transmembrane domain of a CAR include at least the transmembrane region(s) of the alpha, beta or zeta chain of the T-cell receptor, CD28, CD3 epsilon (CD3), CD45, CD4, CD5, CD8, CD9, CD16, CD22, CD33, CD37, CD64, CD80, CD86, CD134, CD137, CD154, or an erythropoietin receptor. In other embodiments, the transmembrane domain is a recombinant or synthetic domain comprising hydrophobic amino acid residues (e.g., leucine and valine). In some cases, the transmembrane domain includes a phenylalanine, tryptophan and valine at one or both ends of the domain.

The transmembrane domain links the antigen binding domain to the intracellular signaling domain of the CAR. In some embodiments, the nucleic acid encoding the antigen binding domain is operably linked to the nucleic acid encoding the transmembrane domain that is operably linked to the nucleic acid encoding the intracellular signaling domain.

In some embodiments, the intracellular signaling domain of a CAR comprises a signal activation or signal transduction domain. As such, an intracellular signaling domain includes any portion of an intracellular signaling domain of a protein known in the art to be sufficient to transduce or transmit a signal, e.g., an activation signal, or to mediate a cellular response within a cell.

In some embodiments, the nucleic acid encoding the CAR of the present invention is operably linked to a promoter such as a synthetic promoter, a constitutive promoter, or an inducible promoter. Useful constitutive promoters include an ubiquitin C promoter, an elongation factor-1 alpha promoter (EF1α promoter), a CMV promoter, and any other constitutive promoter known to those skilled in the art. Useful inducible promoters are described, e.g., in Ede et al, ACS Synth Biol, 2016, 5(5):395-404, and can include cell-type specific promoters and inducible switch promoters. Illustrative constitutive promoters are described in PLoS One, 2010, 5(8):e12413. In some embodiments, the promoter is the EF1α promoter.

The CARs described herein can be introduced into the HIP cells using a vector such as an expression vector, a viral vector, or a non-viral vector. In some instances, the viral vector is a retroviral vector, an adenoviral vector, or an adeno-associated vector. In some embodiments, the nucleic acid encoding the CAR is introduced into a gene locus such as a safe harbor locus of the cell. In other embodiments, the CAR is introduced into HIP cells using non-viral vectors including, but not limited to, minicircle DNA vectors, nude DNA, liposomes, polymerizers, and molecular conjugates.

At present, there are two ways to accomplish gene incorporation with vectors, i.e., viral systems and non-viral systems. The major vectors for gene therapy in basic research and clinical study are viruses, because of the high transfer efficiency, the relatively short time needed to reach the clinically necessary numbers of cultured T cells and the availability of different viruses with different expression characteristics. Virus vectors include retroviruses (including lentivirus), adenovirus and adeno-associated virus. Among them, the most popular tools for gene delivery are genetically engineered retroviruses (e.g., Hu et al., Pharmacol Rev. 2000; 52(4):493-511). Non-viral vectors including, but not limited to, nude DNA, liposomes, polymerizers, and molecular conjugates can be used to introduce a CAR construct into HIP cells. Minicircle DNA vectors that are free of plasmid bacterial DNA sequences are novel non-viral vectors which can be generated in bacteria from a parental plasmid, and can persistently express transgene with high levels in vivo. Minicircle DNA systems can be used in a clinical setting. Detailed descriptions of minicircle DNA vectors can be found, e.g., in Chen et al., Hum Gene Ther. 2005; 16(1):126-131; Kay et al., Nat Biotechnol. 2010; 28(12):1287-1289.

B. Differentiating HIP Cells into HI-CAR-T Cells

The HIP cells comprising a nucleic acid encoding a CAR can be differentiated into a CAR expressing immune cell such as a CAR T cell using any method recognized by one skilled in the art.

Useful methods for differentiating stem cells to immune cells (e.g., immune stem cells, immune progenitor cells, immune multipotent progenitor cells, pre-T cell progenitor cells, pre-NK cell progenitor cells, T cell progenitor cells, NK cell progenitor cells, T cells, NK cells, NKT cells, and B cells) are described in, for example, US2018/0072992, US2017/0296649, and US2016/0009813.

T cells can be αβ T cells, δγ T cells, helper/regulatory T cells, cytotoxic T cells, progenitor T cells (e.g., a progenitor T cell that is CD34+CD7+CD1a− or CD34+CD7+CD5+CD1a−), naïve T cells, central memory T cells, effector T cells, terminal effector T cells, immature T cells, mature T cells, natural killer T cells, and the like. In other words, T cells can be naïve T cells, naïve central memory T cells (TCM cells), effector memory T cells (TEM cells), and effector memory RA T cells (TEMRA cells). Naïve T cells can express CCR7, CD27, CD28, and CD45RA. Naïve central T cells can express CCR7, CD27, CD28, and CD45RO. Effector memory T cells can express PD1, CD27, CD28, and CD45RO. Effector memory RA T cells can express PD1, CD57, and CD45RA.

In some embodiments, the HIP cells comprising a nucleic acid sequence encoding a CAR are cultured in a culture medium comprising a BMP pathway activator, a WNT pathway activator, a MEK inhibitor, a NOTCH pathway inhibitor, a ROCK inhibitor, a TGFβ receptor/ALK inhibitor, a growth factor, a cytokine, and any combination thereof.

The BMP pathway activator can include, but is not limited to, an activator of BMP-2, an activator of BMP-4, an activator of BMP-5, an activator of BMP-6, an activator of BMP-7, an activator of BMP-8, an analog thereof, and a variant thereof.

The GSK3 inhibitor can include, but is not limited to, CHIR99021, an analog thereof, and a variant thereof. The NOTCH pathway activator can include, but is not limited to, Jag1, Jag2, DLL-1, DLL-3, DLL-4, an analog thereof, and a variant thereof. The ROCK inhibitor can include, but is not limited to, Y27632, Fasudil, AR122-86, Y27632 H-1152, Y-30141, Wf-536, HA-1077, hydroxyl-HA-1077, GSK269962A, SB-772077-B, N-(4-pyridyl)-N′-(2,4,6-trichlorophenyl)urea, 3-(4-pyridyl)-1H-indole, and (R)-(+)-trans-N-(4-pyridyl)-4-(1-aminoethyl)-cyclohexanecarboxamide, other ROCK inhibitors disclosed in U.S. Pat. No. 8,044,201, an analog thereof, and a variant thereof. The growth factor can include, but is not limited to, bFGF, EPO, Flt3L, GM-CSF, IGF, TPO, SCF, VEGF, an analog thereof, and a variant thereof. The cytokine can include, but is not limited to, IL-2, IL-3, IL-6, IL-7, IL-11, IL-15, an analog thereof, and a variant thereof.

In some embodiments, the HIP cells carrying a CAR construct are cultured on feeder cells to promote T cell differentiation. The term “feeder cells” can include cells of a different tissue type and typically a different genome that may act to promote proliferation and/or control differentiation of cells they are cocultured with. Undifferentiated HIP cells can be cocultured with feeder cells that direct differentiation towards a particular tissue type (e.g., T cell or a particular T cell subtype). In some embodiments, murine HIP cells are cultured on OP9 or OP9-DL feeder cells. The murine HIP cells can be cultured on feeder cells for about 15 days or more. In other embodiments, the HIP cells are cultured on feeder cells and then after a specific number of days cultured on without feeder cells. In certain embodiments, the HIP cells are not cultured on feeder cells for differentiation into T cells. In some instances, HIP cells are cultured in a medium that promotes CD3 stimulation, and additionally, CD28 stimulation.

In various embodiments, human HIP cells are cultured on feeder cells such as endothelial progenitor cells derived from human HIP cells. In some embodiments, human T cells derived from HIP cells are cultured on endothelial progenitor cells (EPCs) derived from human HIP cells. The cells can be cultured on feeder cells for about 15 days or more. In other embodiments, the cells are cultured on feeder cells and then after a specific number of days cultured on without feeder cells. In certain embodiments, the human EPCs promote generation of HIP-derived T cells. In various embodiments, the human EPCs promote generation of HIP-derived naïve CD4+ T cells. In certain embodiments, the human EPCs hinder the generation of certain subtypes of HIP-derived T cells such as central memory CD4+ T cells.

In some embodiments, HIP-derived T cells are cultured in simulated microgravity (sμg). In particular embodiments, such T cells are produced by differentiation HIP cells using sμg. Human HIP-derived T cells can be cultured in sμg for at least 72 hours. In some embodiments, human HIP-derived T cells are cultured in sμg for 72 hours to 10 days or more. In some cases, culturing the cells in sμg can be used to generate CD8+ T cells. In some embodiments, sμg increases the amount or percentage of TEMRA CD8+ T cells. In other embodiments, sμg does not increase the amount or percentage of naïve CD8+ T cells.

In some embodiments, HIP-derived T cells are cultured in simulated microgravity (sμg) and in culture media comprising IL-2, IL-7, or a combination of IL-2 and IL-7. In some embodiments, HIP-derived T cells cultured in sμg and in the presence of IL-2 to produce central memory CD8+ T cells. In other embodiments, HIP-derived T cells cultured in sμg and in the presence of IL-7 to produce central memory CD8+ T cells. In yet other embodiments, HIP-derived T cells cultured in sμg and in the presence of IL-2 and IL-7 to produce central memory CD8+ T cells.

Methods of assessing the CAR expressing immune cells derived from the HIP cells include, but are not limited to, immunocytochemistry, flow cytometry, cytokine profiling, T cell activation/stimulation assays, target cell cytotoxicity assays, antigen reactivity assays, and in vivo functional assays using animal models.

C. Method of Using the HI-CART Cells

In some aspects, provided herein is a method of treating cancer in a patient, e.g., a human patient, by administrating a therapeutically effective amount of HIP cell derived CAR-T cells. In some cases, the HIP cell derived CAR-T cells are administered with a therapeutically effective carrier.

An “therapeutically effective amount” includes an amount sufficient to effect a beneficial or desired clinical result upon treatment. A therapeutically effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the antigen-binding fragment administered.

The therapeutic cell treatment can be administered by any methods known in the art, including, but not limited to, intravenous administration, subcutaneous administration, intranodal administration, intratumoral administration, intrathecal administration, intrapleural administration, intraperitoneal administration, and direct administration to the thymus. The therapeutic cells can be administered in a bolus or by continuous perfusion.

Cancer can be selected from the group consisting of a blood cancer, a solid tumor cancer, and a liquid tumor cancer. In some embodiments, the blood cancer is a leukemia, a lymphoma or a myeloma. Tumor cancers include, but are not limited to, glioblastoma, melanoma, neuroblastoma, adenocarcinoma, glioma, soft tissue sarcoma, and various carcinomas (including small cell lung cancer). Suitable carcinomas may include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolarcarcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, malignant melanoma, soft tissue sarcomas and leiomyosarcomas.

### IX. DETAILED DESCRIPTION OF EXEMPLARY EMBODIMENTS

In some embodiments, the mouse hypoimmunogenic pluripotent stem (mouse HIP) cell of the invention comprises a genome modification that eliminates B2M activity, a genome modification that eliminates CIITA activity, an exogenous nucleic acid sequence encoding CD47, and an exogenous nucleic acid sequence encoding a CAR construct. In other embodiments, the mouse hypoimmunogenic pluripotent stem cell also comprises an inducible suicide gene.

In other embodiments, the human hypoimmunogenic pluripotent stem (human HIP) cell of the invention comprises a genome modification that eliminates B2M activity, a genome modification that eliminates CIITA activity, an exogenous nucleic acid sequence encoding CD47, and an exogenous nucleic acid sequence encoding a CAR construct. In other embodiments, the human hypoimmunogenic pluripotent stem cell also comprises an inducible suicide gene.

In particular embodiments, the hypoimmunogenic pluripotent stem cell of the invention comprises a genome modification that eliminates B2M activity, a genome modification that eliminates CIITA activity, an exogenous nucleic acid sequence encoding CD47, an exogenous nucleic acid sequence encoding a CAR construct, and a herpes simplex virus thymidine kinase (HSV-tk) gene. In some embodiments, the hypoimmunogenic pluripotent stem cell of the invention comprises a genome modification that eliminates B2M activity, a genome modification that eliminates CIITA activity, an exogenous nucleic acid sequence encoding CD47, an exogenous nucleic acid sequence encoding a CAR construct, and an Escherichia coli cytosine deaminase (CD) gene. In certain embodiments, the hypoimmunogenic pluripotent stem cell of the invention comprises a genome modification that eliminates B2M activity, a genome modification that eliminates CIITA activity, an exogenous nucleic acid sequence encoding CD47, an exogenous nucleic acid sequence encoding a CAR construct, and an exogenous gene encodes an inducible caspase 9 protein.

In various embodiments, the mouse CAR-T cell of the invention is generated from a mouse hypoimmunogenic pluripotent stem cell comprises a genome modification that eliminates B2M activity, a genome modification that eliminates CIITA activity, an exogenous nucleic acid sequence encoding CD47, and an exogenous nucleic acid sequence encoding a CAR construct. In other embodiments, the mouse hypoimmunogenic pluripotent stem cell also comprises an inducible suicide gene. As such, the mouse CAR-T cell has reduced or lacks Major Histocompatibility Antigen Complex I (MHC I) and Major Histocompatibility Antigen Complex II (MHC II) function and overexpresses CD47 protein. The mouse CAR-T cell can be less susceptible to killing by NK cells.

In other embodiments, the human CAR-T cell of the invention is generated from a human hypoimmunogenic pluripotent stem cell comprises a genome modification that eliminates B2M activity, a genome modification that eliminates CIITA activity, an exogenous nucleic acid sequence encoding human CD47, and an exogenous nucleic acid sequence encoding a CAR construct. In other embodiments, the human hypoimmunogenic pluripotent stem cell also comprises an inducible suicide gene. Thus, the human CAR-T cell has reduced or lacks HLA-I and HLA-II function and overexpresses CD47 protein. In some embodiments, the human CAR-T cell has reduced or lacks expression of HLA-A, HLA-B, or HLA-C, has reduced or lacks expression of HLA-DP, HLA-DR, or HLA-DQ protein, and overexpresses human CD47 protein. The human CAR-T cell can be less susceptible to killing by NK cells.

In some embodiments, the human CAR-T cell of the invention is generated from a human hypoimmunogenic pluripotent stem cell comprises a genome modification that eliminates B2M activity, a genome modification that eliminates CIITA activity, an exogenous nucleic acid sequence encoding human CD47, and an exogenous nucleic acid sequence encoding an anti-CD19 CAR construct.

### X. EXAMPLES

### Example 1: Generation of Mouse Induced Pluripotent Stem Cells

The method described herein is adapted from Diecke et al., Sci Rep, 2015, 8081.

Murine tail tip fibroblasts of mice were dissociated and isolated with collagenase type IV (Life Technologies, Grand Island, N.Y., USA) and maintained with Dulbecco's modified Eagle medium (DMEM) containing 10% fetal bovine serum (FBS), L-glutamine, 4.5 g/L glucose, 100 U/mL penicillin, and 100 μg/mL streptomycin at 37° C., 20% O2, and 5% CO2 in a humidified incubator.

1×10⁶ murine fibroblasts were then reprogrammed using a novel codon optimized mini-intronic plasmid (co-MIP) (10-12 μm of DNA) expressing the four reprogramming factors Oct4, KLF4, Sox2 and c-Myc using the Neon Transfection system. After transfection, fibroblasts were plated on a murine embryonic fibroblasts (MEF) feeder layer and kept in fibroblast media with the addition of sodium butyrate (0.2 mM) and 50 μg/mL ascorbic acid.

When ESC-like colonies appeared, media was changed to murine iPSC media containing DMEM, 20% FBS, L-glutamine, non-essential amino acids (NEAA), β-mercaptoethanol, and 10 ng/mL leukemia inhibitory factor (LIF). After 2 passages, the murine iPSCs were transferred to 0.2% gelatin coated plates and further expanded. With every passage, the iPSCs were sorted for the murine pluripotency marker SSEA-1 using magnetic activated cell sorting (MACS).

The isolated mouse iPSCs can be used to generate mouse hypoimmunogenic iPSCs according to the method described above.

### Example 2: Generation of Human Induced Pluripotent Stem Cells

The Gibco™ Human Episomal iPSC Line (catalog number A18945, ThermoFisher) was derived from CD34+ cord blood using a three-plasmid, seven-factor (SOKMNLT; SOX2, OCT4 (POU5F1), KLF4, MYC, NANOG, LIN28, and SV40L T antigen) EBNA-based episomal system. This iPSC line is considered to be zero foot-print as there was no integration into the genome from the reprogramming event. It has been shown to be free of all reprogramming genes. Protocols for thawing, culturing, and passaging the human iPSCs are provided in the product manual.

Pluripotency of the human iPSCs can be determined by in vivo teratoma assays and in vitro pluripotent gene expression assays (e.g., PCR and arrays) or by fluorescence staining for pluripotent markers.

The Gibco™ Human Episomal iPSC Line has a normal karyotype and endogenous expression of pluripotent markers like OCT4, SOX2, and NANOG (as shown by RT-PCR) and OCT4, SSEA4, TRA-1-60 and TRA-1-81 (as shown by ICC). Whole genome expression and epigenetic profiling analyses demonstrated that this episomal hiPSC line is molecularly indistinguishable from human embryonic stem cell lines (Quintanilla et al., PloS One, 2014, 9(1): e85419). In directed differentiation and teratoma analyses, these hiPSCs retained their differentiation potential for the ectodermal, endodermal, and mesodermal lineages (Burridge et al., PLoS One, 2011, 6(4): e18293). In addition, vascular, hematopoietic, neural, and cardiac lineages were derived with robust efficiencies (Burridge et al., supra).

TABLE 1
Illustrative protocol for culturing human iPSCs (e.g., Cas9 iPSCs)

| Title | Human iPSC culture | |
|---|---|---|
| Introduction | Induced Pluripotent stem cells have the capacity to give rise to differentiated | |
| | progeny representative of all three germ layers (ectoderm, endoderm, and | |
| | mesoderm). The ability to expand pluripotent cells in vitro and subject them to | |
| | direct differentiation to produce specific cell types is crucial to the development of | |
| | cell-based therapies to replace or restore tissue that has been damaged by disease | |
| | or injury. | |
| Materials | 1. Essential 8 Flex Media (Thermo Fisher Scientific, cat.no. A2858501) | |
| | 2. Revita Cell Supplement (Thermo Fisher Scientific, cat.no. A2644501) | |
| | 3. diluted Matrigel (Corning, cat.no. 356231), diluted in Knockout DMEM | |
| | (Thermo Fisher Scientific, cat.no. 10829) | |
| | 4. Versene (Thermo Fisher Scientific, cat.no.15040066) | |
| | 5. 10 cm² cell culture plates (Corning, cat.no.353003) | |
| Protocol | | Notes |
| 1. | Thaw 1x vial in 1x 10 cm² dish, after approx. 4-5 days, the | |
| | cells will reach 60-70% confluency and are ready for | |
| | splitting | |
| 2. | Reconstitute Matrigel 1:40 in cold Knockout DMEM and | |
| | mix well. Place dishes in 37° C. incubator for 30 minutes to | |
| | use plates immediately or seal with parafilm and store at 2- | |
| | 8° C. for up to 7 days. | |
| 3. | Culture Cas9 human iPSC on diluted Matrigel (1:40 in KO | Incubate cells at |
| | DMEM) coated 10 cm2 dishes in Essential 8 Flex Media. | 37° C./ 5% CO₂. |
| 4. | Change media daily and passage the cells every 3-4 days | Pipette gently. Do not |
| | 1:6 using Versene for 9 min at 37° C. | vortex!! Centrifugation |
| | | 800 rpm, 4 min at 4° C. |
| 5. | Revita Cell Supplement was added 1:100 in the media | |
| | after splitting for the first 24h. | |

The isolated human iPSCs can be used to generate human hypoimmunogenic iPSCs according to the method described above.

### Example 3: Hypoimmunogenic Pluripotent Cells were Less Susceptible to NK Cell Killing and Macrophage Phagocytosis

Examples were performed to evaluate the ability of hypoimmunogenic pluripotent cells (e.g., mouse b2m−/−ciita−/−CD47 tg iPSCs and human B2M−/−CIITA−/− CD47 tg iPSCs) and to evade the immune innate response pathways.

In particular, enzyme-linked immunospot (Elispot) assays were performed. NK cells were co-cultured with mouse HIP cells or human HIP cells (mouse B2m−/−Ciita−/−CD47 tg iPSCs or human B2M−/−CIITA−/−CD47 tg iPSCs) and IFNγ release was measured (e.g., innate IFNγ spot frequencies were measured using an Elispot plate reader). In some examples, CD47 was blocked by using an anti-CD47 antibody.

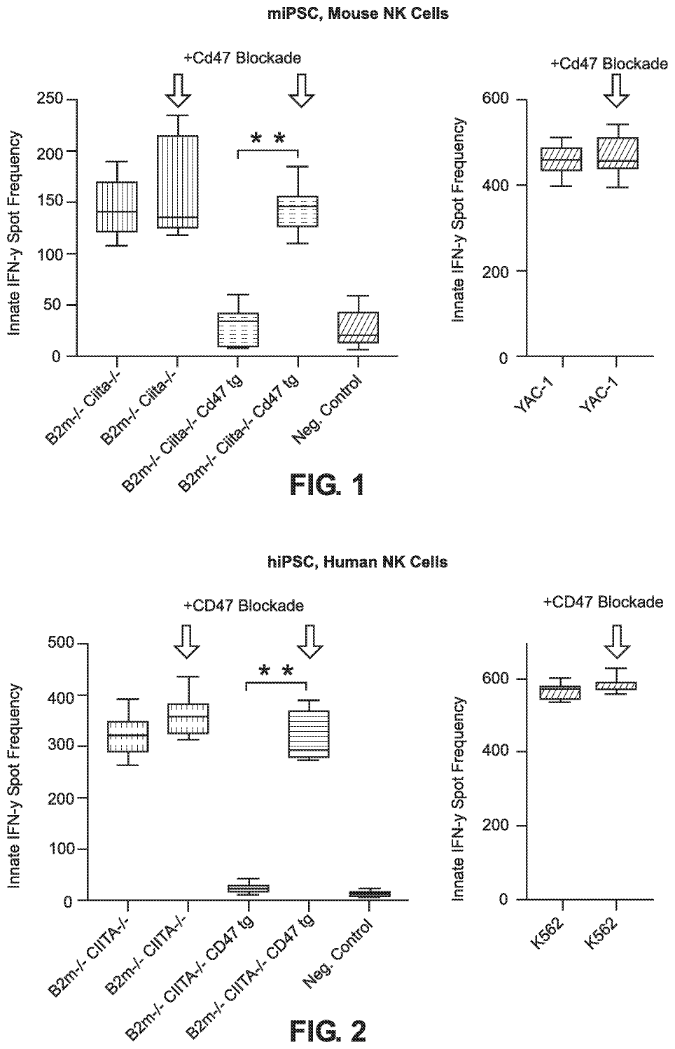

Mouse B2m−/−Ciita−/−CD47 tg iPSCs co-cultured with mouse NK cells such as approximately 95% NK cells and 5% macrophages failed to stimulate NK cell activation (FIG. 1). Mouse B2m−/−Ciita−/− iPSCs triggered IFNγ release by NK cells in the Elispot assay, while mouse B2m−/−Ciita−/−CD47 tg iPSCs did not. Blocking CD47 (e.g., use of an anti-CD47 antibody) had no effect on the mouse B2m−/−Ciita−/− iPSCs. However, CD47 blockage fully abolished the protection B2m−/−Ciita−/−CD47 tg iPSCs had. YAC-1 cells which are known to activate NK cells and thus release of IFNγ served as a control.

Human B2M−/−CIITA−/−CD47 tg iPSCs co-cultured with human NK cells also failed to stimulate NK cell activation. FIG. 2 shows that human B2M−/−CIITA−/− iPSCs triggered IFNγ release by NK cells in the Elispot assay, while human B2M−/−CIITA−/−CD47 tg iPSCs did not. Blockage of CD47 had no effect on human B2M−/−CIITA−/− iPSCs, but it did abolish the protection human B2M−/−CIITA−/−CD47 tg iPSCs had. K562 cells which are known to activate NK cells and thus release of IFNγ served as a control.

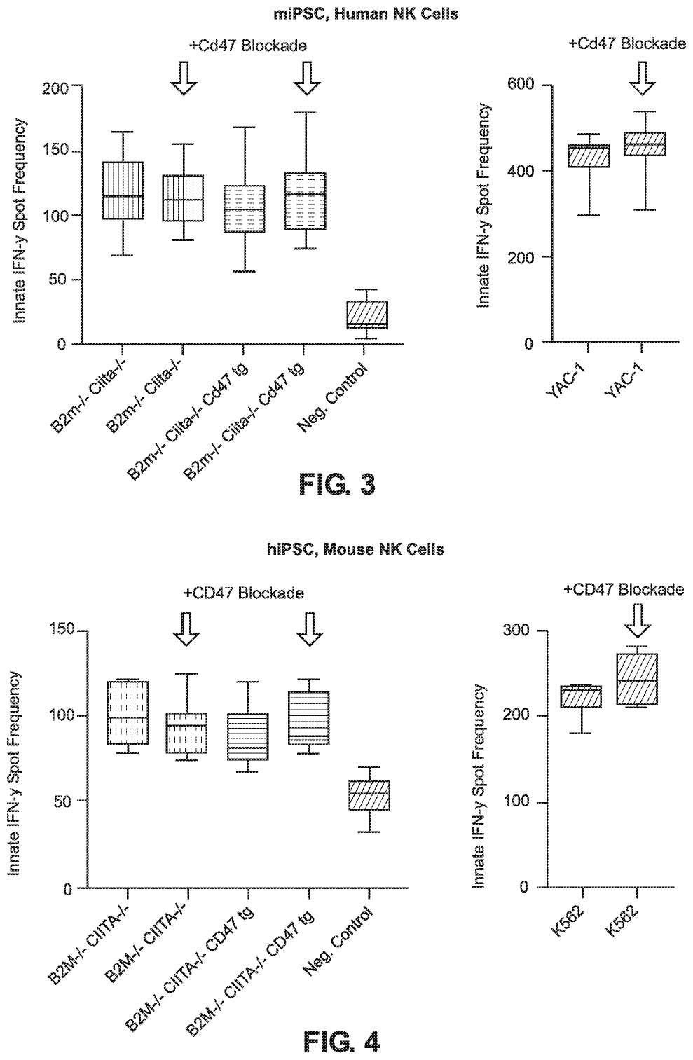

FIG. 3 shows Elispot results of mouse B2m−/−Ciita−/−CD47 tg iPSCs incubated with human NK cells (approximately 95% NK cells and 5% macrophages). Mouse B2m−/− Ciita−/− iPSCs and mouse B2m−/−Ciita−/−CD47 tg iPSCs triggered IFNγ release by human NK cells. Blockage of CD47 had not effect on the NK cell response. YAC-1 cells elicited a strong IFNγ release by human NK cells and served as a control.

FIG. 4 shows Elispot results of human B2M−/−CIITA−/−CD47 tg iPSCs incubated with mouse NK cells (approximately 95% NK cells and 5% macrophages). Human B2M−/−CIITA−/− iPSCs and human B2M−/−CIITA−/−CD47 tg iPSCs triggered IFNγ release by mouse NK cells. Blockage of CD47 had not effect on the NK cell response. Human K562 cells elicited a strong IFNγ release by mouse NK cells and served as a control.

Macrophage phagocytosis assays were also performed to determine if the HIP cells of the present invention are susceptible to macrophage phagocytosis. Briefly, HIP cells described herein were labeled with firefly luciferase and co-cultured with macrophages. The viability or presence of the HIP cells was analyzed by a luciferase reporter assay.

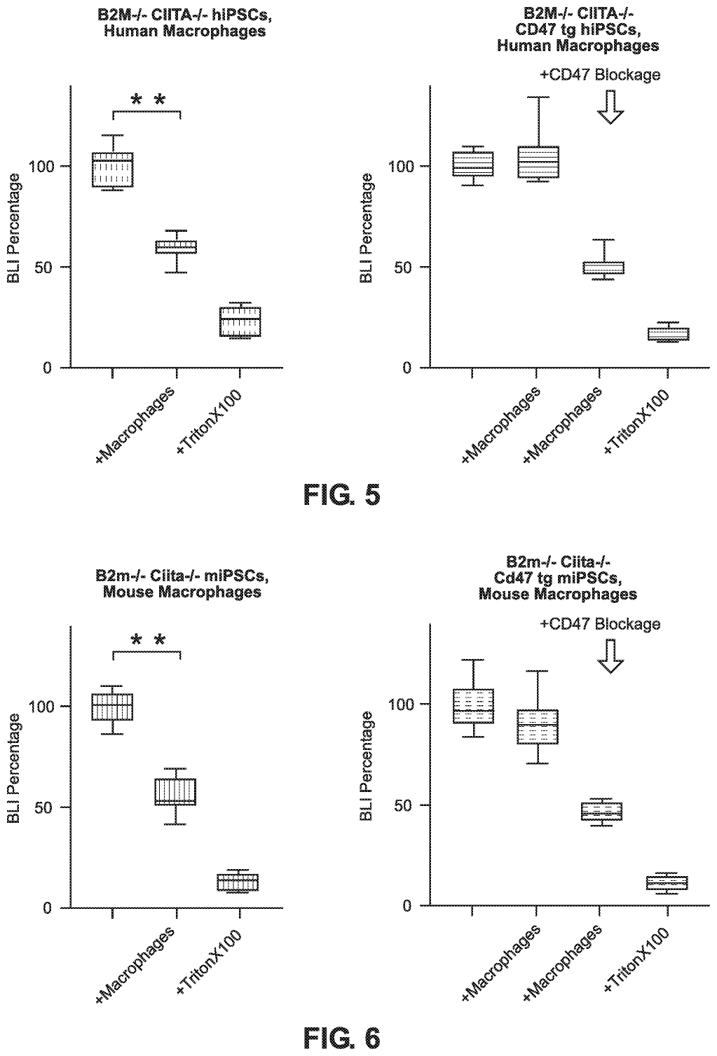

FIG. 5 shows phagocytosis assay results of firefly luciferase labeled human B2M−/−CIITA−/−CD47 tg iPSCs co-cultured with human macrophages. The viability signal of the human B2M−/−CIITA−/− iPSCs significantly dropped when incubated with macrophages. On the other hand, the viability signal of the human B2M−/−CIITA−/−CD47 tg iPSCs did not change in the presence of human macrophages. TritonX-100 which killed all HIP cells was used as a control. Blockage of CD47 eliminated the protective features of human B2M−/−CIITA−/−CD47 tg iPSCs and made them susceptible to macrophage phagocytosis or elimination.

FIG. 6 shows phagocytosis assay results of firefly luciferase labeled mouse B2m−/−Ciita−/−CD47 tg iPSCs co-cultured with mouse macrophages.

The viability signal of the mouse B2m−/−Ciita−/− iPSCs significantly dropped when incubated with macrophages. In contrast, the viability signal of the mouse B2m−/− Ciita−/−CD47 tg iPSCs did not change in the presence of mouse macrophages. TritonX-100 which killed all HIP cells was used as a control. Blockage of CD47 eliminated the protective features of mouse B2M−/−CIITA−/−CD47 tg iPSCs and made them susceptible to macrophage phagocytosis or elimination. TritonX-100 which killed all HIP cells was used as a control.

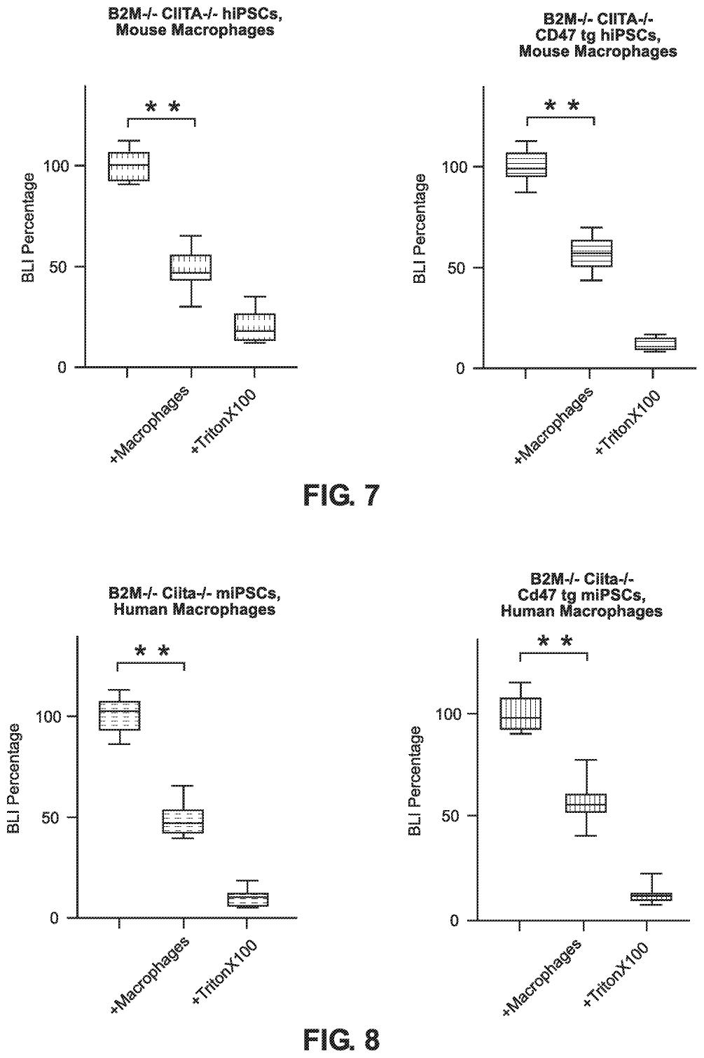

FIG. 7 shows phagocytosis assay results of firefly luciferase labeled human B2M−/−CIITA−/−CD47 tg iPSCs co-cultured with mouse macrophages. The viability signals of both human B2M−/−CIITA−/− iPSCs and human B2M−/−CIITA−/−CD47 tg iPSCs dropped significantly when co-cultured with mouse macrophages. TritonX-100 which killed all HIP cells was used as a control.

FIG. 8 shows phagocytosis assay results of firefly luciferase labeled mouse B2m−/−Ciita−/−CD47 tg iPSCs co-cultured with human macrophages. The viability signals of both mouse B2m−/−Ciita−/− iPSCs and mouse B2m−/−Ciita−/−CD47 tg iPSCs dropped significantly when co-cultured with human macrophages. TritonX-100 which killed all HIP cells was used as a control.

The results provided herein show that mouse B2m−/−Ciita−/−CD47 tg iPSCs and human B2M−/−CIITA−/−CD47 tg iPSCs were able to evade innate immune responses, such as NK cell activation and macrophage phagocytosis.

### Example 4: Generation of T Cells from HIP Cells

This example shows that HIP cells (e.g., mouse HIP cells and human HIP cells) were differentiated into T cells including CD8+ low, CD8+ high, CD4+, CD4+/CD8+ high, and CD4+/CD8+ low T cells. The example also shows that the stimulatory signals and cytokines were used to direct differentiation into different T cell subtypes. It was shown that endothelial progenitor cells (EPCs) such as HIP-derived EPCs were used to increase the number of naïve CD4+ T cells and decrease the number of central memory CD4+ T cells. This example also demonstrates that simulated microgravity (sμg) stimulation alone or in combination with cytokines (e.g., IL-2, IL-7, or a combination of IL-2 and IL-2) induced differentiation of HIP derived T cells into central memory CD8+ T cells.

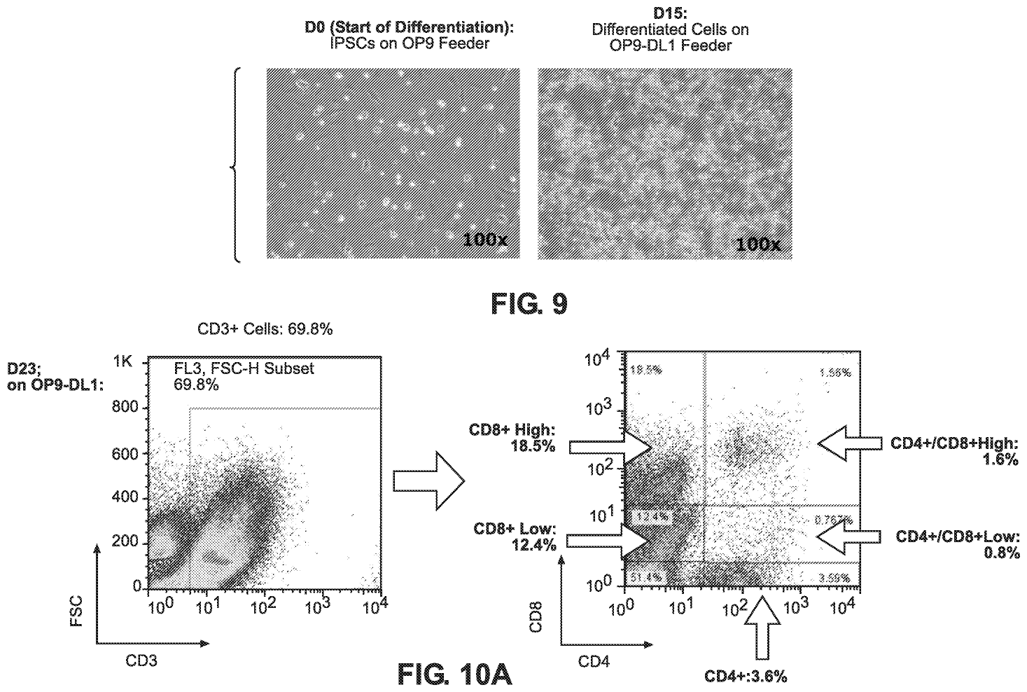

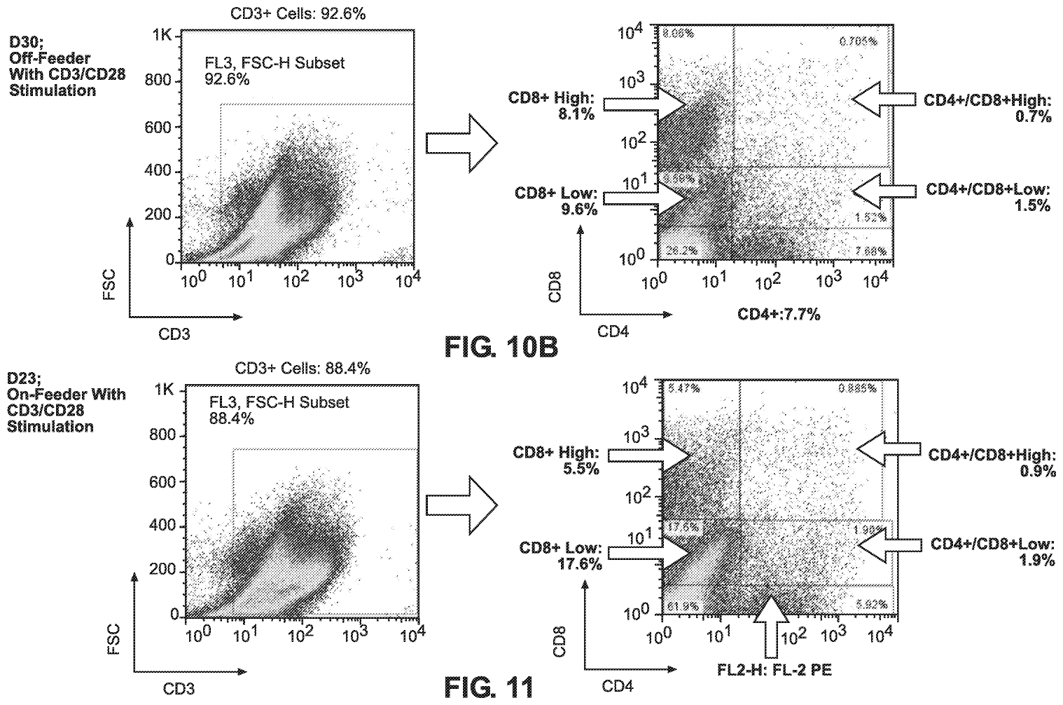

Mouse HIPs cells were cultured on OP9 cells at DO (the start of differentiation). On D15 of differentiation on OP9-DL1 feeder cells, the resulting cells resembled T cells (FIG. 9). FACS analysis shows that on D23 the mouse HIP cells cultured on OP9-DL1 differentiated into CD3+ T cells (69.8%), CD8+ high T cells (18.5%), CD8+low T cells (12.4%), CD4+ T cells (3.6%), CD4+/CD8+ high T cells (1.6%), and CD4+/CD8+ low T cells (0.8%) (FIG. 10A). FACS analysis also shows that on D30 the mouse HIP cells cultured off feeder cells and in the presence of CD3 and CD28 stimulation differentiated into CD3+ T cells (92.6%), CD8+ high T cells (8.1%), CD8+ low T cells (9.6%), CD4+ T cells (7.7%), CD4+/CD8+ high T cells (0.7%), and CD4+/CD8+ low T cells (1.5%) (FIG. 10B). FACS analysis also shows that on D23 the mouse HIP cells cultured on feeder cells (e.g., OP9-DL1 cells) and in the presence of CD3 and CD28 stimulation differentiated into CD3+ T cells (88.4%), CD8+ high T cells (5.5%), CD8+ low T cells (17.6%), CD4+ T cells (5.9%), CD4+/CD8+ high T cells (0.9%), and CD4+/CD8+ low T cells (1.9%) (FIG. 11). The results show that mouse HIP cells were differentiated into T cells and that particular T cell subtypes can be obtained by using different stimulatory signals and cytokines, such as CD3, CD28, IL-2, IL-15, and IL-7. Under these conditions, the percentage of HIP-derived CD4+ T cells remained low compared to the percentage of CD3+ cells and CD8+ cells.

T cells can be naïve T cells, naïve central memory T cells (TCM cells), effector memory T cells (TEM cells), and effector memory RA T cells (TEMRA cells). Naïve T cells can express CCR7, CD27, CD28, and CD45RA. Naïve central T cells can express CCR7, CD27, CD28, and CD45RO. Effector memory T cells can express PD1, CD27, CD28, and CD45RO. Effector memory RA T cells can express PD1, CD57, and CD45RA.

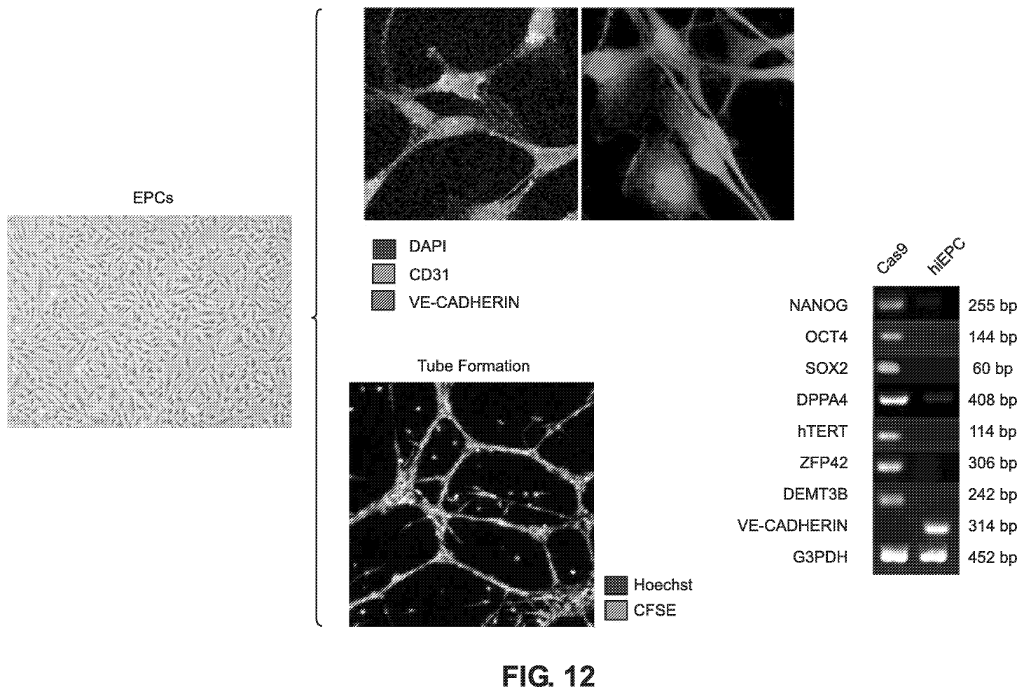

Examples were performed to generate CD4+ T cells differentiated from human HIP cells. It was hypothesized that co-culturing T cells derived from human HIP cells with endothelial progenitor cells (EPCs) derived from HIP cells could increase the number of HIP derived CD4+ T cells. FIG. 12 provides images of EPCs derived from human HIP cells. In some embodiments, EPCs were produced by differentiating human HIP cells in media comprising one or more of the following factors: bFGF, VEGF, FGF, Rock inhibitor (e.g., Y-27632), TGFβ pathway inhibitor (e.g., SB-431542), GSK3 inhibitor (CHIR-99021), or any combination thereof. Co-culturing of human EPCs and human T cells derived from human HIP cells increased the number of CD4+ T cells (FIG. 13A) compared the absence of human EPCs. FIG. 13B shows that co-culture with human HIP derived EPCs induced differentiation into naïve CD45RA+CCR7+CD4+ T cells. FIG. 13C shows that co-culture with human HIP derived EPCs prevented differentiation into central memory CD45RA−CCR7+CD4+ T cells. This study illustrates that CD4+ T cell differentiation was increased by co-culturing with HIP-derived endothelial progenitor cells. Co-culturing with EPCs increased the number of naïve CD4+ T cells derived from human HIP cells and decreased the number of central memory CD4+ T cells.

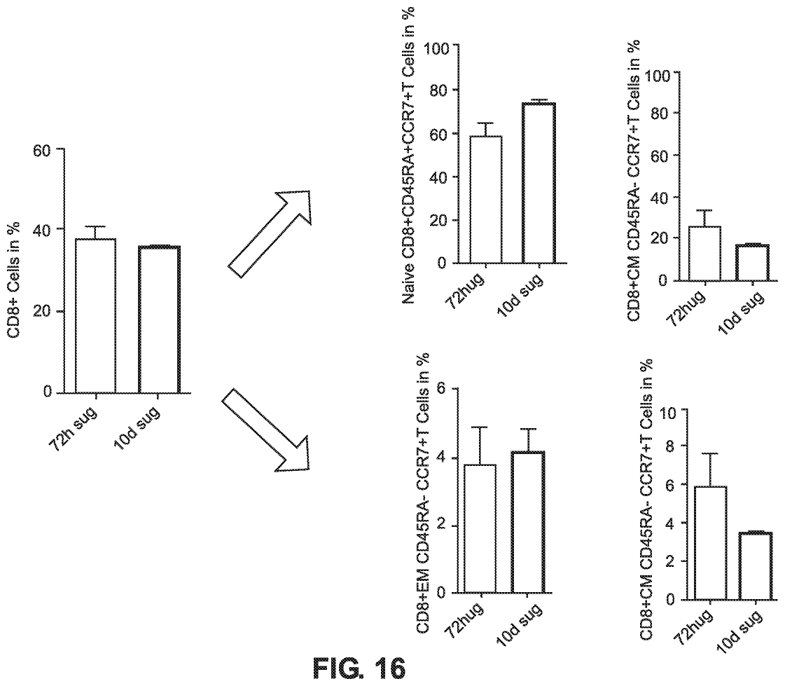

Additional examples were performed to develop a novel method for generating specific T cell subtypes by way of T cell differentiation of HIP cells. The examples evaluated the effect of using simulated microgravity (sμg) on the resulting T cells. Sig can be produced using a Random Positioning Machine (Airbus) or a similar system that rotates, such as but not limited to Synthecon's stem cell culture system with a rotator base. Human HIP derived T cells were cultured for 72 hours under sμg conditions. As a control, the cells were cultured for 72 hours at 1 g (standard gravity). FIG. 14A shows that the morphology of the T cells cultured at sμg was different than those cultured at 1 g (1 gravity). The viability of the T cells was not different between the sμg condition and the standard condition. Analysis of the CD8+ T cells showed that simulated microgravity produced fewer CD8+ T cells and fewer naïve CD8+(CD8+CD45RA+CCR7+) T cells (FIG. 15). Simulated microgravity also increased the number of TEMRA CD8+(CD8+CD45RA+CCR7-) cells compared to standard culture conditions (FIG. 15). FIG. 16 shows that increasing the incubation time of the sμg did not provide a beneficial effect. Sμg for 72 hours is sufficient and a longer exposure of 10 days did not significantly increase the number of CD8+ T cells or different subtypes of CD8+ T cells.

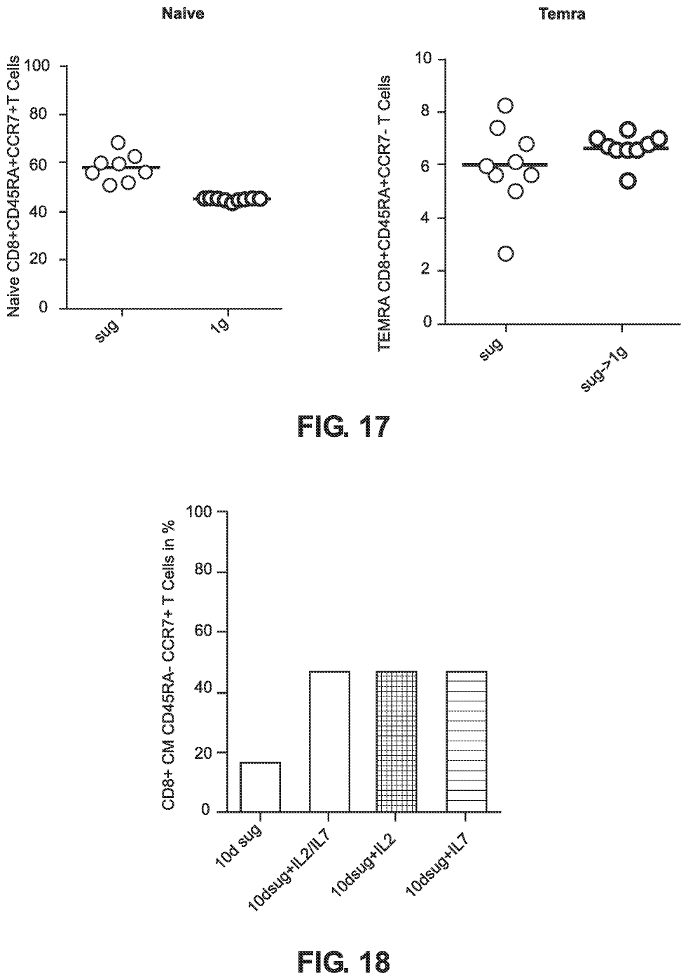

It was also analyzed whether culturing HIP-derived human T cells in sμg for 72 hours and then at 1 g for another 72 hours affected T cell differentiation. The results showed that T cell differentiation using sμg was not reversible. Treatment in sμg and at 1 g did not have a significant effect on the differentiation compared to sμg alone (FIG. 17).

It was also shown that differentiating HIP-derived human T cells in sμg and in the presence of one or more cytokines induced the generation of central memory CD8+ (CD8+CD45RA−CCR7+) T cells. FIG. 18 shows that central memory CD8+ T cells were induced when the cells were cultured in sμg for 10 days and with IL-2, IL-7, or a combination of IL-2 and IL-7.

This example clearly shows that culturing HIP-derived human T cells in simulated microgravity for 72 hours decreased the number of naïve CD8+ T cells produced. Such culture conditions increased the number of CD8+ TEMRA cells. The resulting T cells were viable after 72 hours in sμg. When combined with cytokine stimulation, sμg culturing increased the number of CD8+CM T cells.

The example provides data showing that mouse and human HIP cells were differentiated into T cells. Certain T cell subtypes were induced using particular culturing conditions. HIP cells were differentiated into T cells using feeder cells, and optionally CD3 and CD28 stimulation. HIP-derived human T cells were co-cultured with HIP-derived endothelial cells to generate HIP-derived CD4+ T cells. In some cases, such HIP-derived CD4+ T cells were CD4+ naïve T cells. HIP-derived human T cells were cultured in sμg for at least 72 hours to generate TEMRA CD8+ T cells. HIP-derived human T cells were cultured in sμg and stimulated with cytokines for at least 72 hours (e.g., 10 days) to generate central memory CD8+ T cells. The methods described herein can be used to obtain specific T cell populations that can be applicable to CAR technology. The methods can also be utilized for pluripotent stem cell-derived T cell differentiation, hematopoetic stem cell-derived T cell differentiation, and differentiation of other immune cell populations.

All publications and patent documents disclosed or referred to herein are incorporated by reference in their entirety. The foregoing description has been presented only for purposes of illustration and description. This description is not intended to limit the invention to the precise form disclosed.

It is intended that the scope of the invention be defined by the claims appended hereto.

Informal Sequence Listing
Human β-2-Microglobulin protein
SEQ ID NO: 1
MSRSVALAVLALLSLSGLEAIQRTPKIQVYSRHPAENGKSNFLNCYVSGF
HPSDIEVDLLKNGERIEKVEHSDLSFSKDWSFYLLYYTEFTPTEKDEYAC
RVNHVTLSQPKIVKWDRDI
Human CIITA protein, 160 amino acid N-terminus
SEQ ID NO: 2
MRCLAPRPAGSYLSEPQGSSQCATMELGPLEGGYLELLNSDADPLCLYHF
YDQMDLAGEEEIELYSEPDTDTINCDQFSRLLCDMEGDEETREAYANIAE
LDQYVFQDSQLEGLSKDIFKHIGPDEVIGESMEMPAEVGQKSQKRPFPEE
LPADLKHWKP
Human CD47 protein
SEQ ID NO: 3
MWPLVAALLLGSACCGSAQLLFNKTKSVEFTFCNDTVVIPCFVTNMEAQN
TTEVYVKWKFKGRDIYTFDGALNKSTVPTDFSSAKIEVSQLLKGDASLKM
DKSDAVSHTGNYTCEVTELTREGETIIELKYRVVSWFSPNENILIVIFPI
FAILLFWGQFGIKTLKYRSGGMDEKTIALLVAGLVITVIVIVGAILFVPG
EYSLKNATGLGLIVTSTGILILLHYYVFSTAIGLTSFVIAILVIQVIAYI
LAVVGLSLCIAACIPMHGPLLISGLSILALAQLLGLVYMKFVE
Herpes Simplex Virus Thimidine Kinase (HSV-tk)
protein
SEQ ID NO: 4
MASYPCHQHASAFDQAARSRGHSNRRTALRPRRQQEATEVRLEQKMPTLL
RVYIDGPHGMGKTTTTQLLVALGSRDDIVYVPEPMTYWQVLGASETIANI
YTTQHRLDQGEISAGDAAVVMTSAQITMGMPYAVTDAVLAPHVGGEAGSS
HAPPPALTLIFDRHPIAALLCYPAARYLMGSMTPQAVLAFVALIPPTLPG
TNIVLGALPEDRHIDRLAKRQRPGERLDLAMLAAIRRVYGLLANTVRYLQ
GGGSWWEDWGQLSGTAVPPQGAEPQSNAGPRPHIGDTLFTLFRAPELLAP
NGDLYNVFAWALDVLAKRLRPMHVFILDYDQSPAGCRDALLQLTSGMVQT
HVTTPGSIPTICDLARTFAREMGEAN
Escherichia coli Cytosine Deaminase (CD) protein
SEQ ID NO: 5
MSNNALQTIINARLPGEEGLWQIHLQDGKISAIDAQSGVMPITENSLDAE
QGLVIPPFVEPHIHLDTTQTAGQPNWNQSGTLFEGIERWAERKALLTHDD
VKQRAWQTLKWQIANGIQHVRTHVDVSDATLTALKAMLEVKQEVAPWIDL
QIVAFPQEGILSYPNGEALLEEALRLGADVVGAIPHFEFTREYGVESLHK
TFALAQKYDRLIDVHCDEIDDEQSRFVETVAALAHHEGMGARVTASHTTA
MHSYNGAYTSRLFRLLKMSGINFVANPLVNIHLQGRFDTYPKRRGITRVK
EMLESGINVCFGHDDVFDPWYPLGTANMLQVLHMGLHVCQLMGYGQINDG
LNLITHHSARTLNLQDYGIAAGNSANLIILPAENGFDALRRQVPVRYSVR
GGKVIASTQPAQTTVYLEQPEAIDYKR
Truncated human Caspase 9 protein
SEQ ID NO: 6
GFGDVGALESLRGNADLAYILSMEPCGHCLIINNVNFCRESGLRTRTGSN
IDCEKLRRRFSSLHFMVEVKGDLTAKKMVLALLELAQQDHGALDCCVVVI
LSHGCQASHLQFPGAVYGTDGCPVSVEKIVNIFNGTSCPSLGGKPKLFFI
QACGGEQKDHGFEVASTSPEDESPGSNPEPDATPFQEGLRTFDQLDAISS
LPTPSDIFVSYSTFPGFVSWRDPKSGSWYVETLDDIFEQWAHSEDLQSLL
LRVANAVSVKGIYKQMPGCFNFLRKKLFFKTS

What is claimed is:

1. An isolated hypoimmunogenic induced pluripotent stem (HIP) cell comprising a nucleic acid encoding a chimeric antigen receptor (CAR),
wherein endogenous β-2 microglobulin (B2M) gene activity and endogenous class II transactivator (CIITA) gene activity have been eliminated and CD47 expression has been increased.

2. The isolated HIP cell of claim 1, wherein the CAR comprises an extracellular domain, a transmembrane domain, and an intracellular signaling domain.

3. The isolated HIP cell of claim 2, wherein the extracellular domain binds to an antigen selected from the group consisting of CD19, CD20, CD22, CD38, CD123, CD171, CS1, BCMA, MUC16, ROR1, and WT1.

4. The isolated HIP cell of claim 2 or 3, wherein the extracellular domain comprises a single chain variable fragment (scFv).

5. The isolated HIP cell of any one of claims 2 to 4, wherein the transmembrane domain comprises CD3ζ, CD4, CD8α, CD28, 4-1BB, OX40, ICOS, CTLA-4, PD-1, LAG-3, and BTLA.

6. The isolated HIP cell of any one of claims 2 to 5, wherein the intracellular signaling domain comprises CD3ζ, CD28, 4-1BB, OX40, ICOS, CTLA-4, PD-1, LAG-3, and BTLA.

7. The isolated HIP cell of any one of claims 2 to 6, wherein the nucleic acid encoding the CAR is introduced into the iPSC after B2M gene activity and CIITA gene have been eliminated and CD47 expression has been increased.

8. The isolated HIP cell of any one of claims 1 to 7, wherein the HIP cell is a human induced pluripotent stem cell, the B2M gene is human B2M gene, the CIITA gene is human B2M gene, and the increased CD47 expression results from introducing into the iPSC at least one copy of a human CD47 gene under the control of a promoter.

9. The isolated HIP cell of any one of claims 1 to 7, wherein the HIP cell is a mouse induced pluripotent stem cell, the B2M gene is mouse B2M gene, the CIITA gene is mouse B2M gene, and the increased CD47 expression results from introducing into the iPSC at least one copy of a mouse CD47 gene under the control of a promoter.

10. The isolated HIP cell of claim 8 or 9, wherein the promoter is a constitutive promoter.

11. The isolated HIP cell of any one of claims 1 to 10, wherein the elimination of B2M gene activity results from a Clustered Regularly Interspaced Short Palindromic Repeats (CRISPR)/Cas9 reaction that disrupts both alleles of the B2M gene.

12. The isolated HIP cell of any one of claims 1 to 11, wherein the elimination of CIITA gene activity results from a CRISPR/Cas9 reaction that disrupts both alleles of the CIITA gene.

13. The isolated HIP cell of any one of claims 1 to 12, further comprising a suicide gene that is activated by a trigger agent that induces the hypoimmunogenic pluripotent cell to die.

14. The isolated HIP cell of claim 13, wherein the suicide gene is a herpes simplex virus thymidine kinase (HSV-tk) gene and the trigger agent is ganciclovir.

15. The isolated HIP cell of claim 14, wherein the HSV-tk gene encodes a protein comprising at least 90% sequence identity to SEQ ID NO:4.

16. The isolated HIP cell of claim 14, wherein the HSV-tk gene encodes a protein comprising the amino acid sequence of SEQ ID NO:4.

17. The isolated HIP cell of claim 13, wherein the suicide gene is an Escherichia coli cytosine deaminase (CD) gene and the trigger agent is 5-fluorocytosine (5-FC).

18. The isolated HIP cell of claim 17, wherein the CD gene encodes a protein comprising at least 90% sequence identity to SEQ ID NO:5.

19. The isolated HIP cell of claim 17, wherein the CD gene encodes a protein comprising the amino acid sequence of SEQ ID NO:5.

20. The isolated HIP cell of claim 13, wherein the suicide gene encodes an inducible caspase 9 protein and the trigger agent is a chemical inducer of dimerization (CID).

21. The isolated HIP cell of claim 20, wherein the inducible caspase 9 protein comprises at least 90% sequence identity to SEQ ID NO:6.

22. The isolated HIP cell of claim 20, wherein the inducible caspase 9 protein comprises the amino acid sequence of SEQ ID NO:6.

23. The isolated HIP cell of any one of claims 20 to 22, wherein the CID is compound AP1903.

24. An isolated hypoimmune CAR-T cell produced by in vitro differentiation of the HIP cell of any one of claims 1 to 23.

25. The isolated hypoimmune CAR-T cell of claim 24, wherein the CAR-T cell is a hypoimmune cytotoxic CAR-T cell.

26. The isolated hypoimmune CAR-T cell of claim 24 or 25, wherein the in vitro differentiation comprises culturing the HIP cell in a culture media comprising one or more growth factors or cytokines selected from the group consisting of bFGF, EPO, Flt3L, IGF, IL-2, IL-3, IL-6, IL-7, IL-15, GM-CSF, SCF, and VEGF.

27. The isolated hypoimmune CAR-T cell of any one of claims 24 to 26, wherein the culture media further comprises one or more selected from the group consisting of a BMP activator, a GSK3 inhibitor, a ROCK inhibitor, a TGFβ receptor/ALK inhibitor, and a NOTCH activator.

28. The isolated hypoimmune CAR-T cell of any one of claims 24 to 27, wherein the in vitro differentiation comprises culturing the HIP cell on feeder cells.

29. The isolated hypoimmune CAR-T cell of any one of claims 24 to 28, wherein the in vitro differentiation comprises culturing in simulated microgravity.

30. The isolated hypoimmune CAR-T cell of claim 29, wherein the culturing in simulated microgravity is for at least 72 hours.

31. The isolated hypoimmune CAR-T cell of any one of claims 24 to 30 for use as a treatment of cancer.

32. A method of treating a patient with cancer by administering a composition comprising a therapeutically effective amount of the isolated hypoimmune CAR-T cells of any one of claims 24 to 27.

33. The method of claim 32, wherein the composition further comprises a therapeutically effective carrier.

34. The method of claim 32 or 33, wherein the administration comprises intravenous administration, subcutaneous administration, intranodal administration, intratumoral administration, intrathecal administration, intrapleural administration, and intraperitoneal administration.

35. The method of any one of claims 32 to 34, wherein the administration further comprises a bolus or by continuous perfusion.

36. The method of any one of claims 32 to 35, wherein the cancer is a blood cancer selected from the group consisting of leukemia, lymphoma, and myeloma.

37. The method of any one of claims 32 to 35, wherein the cancer is a solid tumor cancer or a liquid tumor cancer.

38. A pure population of hypoimmune CAR-T cells derived from a population of isolated HIP cells by a method comprising in vitro differentiation,
wherein the isolated HIP cells comprise a nucleic acid encoding a chimeric antigen receptor (CAR) and a suicide gene that is activated by a trigger agent that can induce the HIP cells to die, and
wherein endogenous β-2 microglobulin (B2M) gene activity and endogenous class II transactivator (CIITA) gene activity have been eliminated and CD47 expression has been increased in the HIP cells.

39. The pure population of isolated hypoimmune CAR-T cells of claim 38, wherein the suicide gene is a herpes simplex virus thymidine kinase (HSV-tk) gene and the trigger agent is ganciclovir, the suicide gene is an Escherichia coli cytosine deaminase (CD) gene and the trigger agent is 5-fluorocytosine (5-FC), or the suicide gene is an inducible caspase 9 protein and the trigger agent is a chemical inducer of dimerization (CID).

40. The pure population of isolated hypoimmune CAR-T cells of claim 38 or 39, wherein the CAR-T cells are hypoimmune cytotoxic CAR-T cells.

41. The pure population of isolated hypoimmune CAR-T cells of any one of claims 38 to 40, wherein the in vitro differentiation comprises culturing the HIP cells in a culture media comprising one or more growth factors or cytokines selected from the group consisting of bFGF, EPO, Flt3L, IGF, IL-2, IL-3, IL-6, IL-7, IL-15, GM-CSF, SCF, and VEGF.

42. The pure population of isolated hypoimmune CAR-T cells of any one of claims 38 to 41, wherein the culture media further comprises one or more selected from the group consisting of a BMP activator, a GSK3 inhibitor, a ROCK inhibitor, a TGFβ receptor/ALK inhibitor, and a NOTCH activator.

43. The pure population of isolated hypoimmune CAR-T cells of any one of claims 38 to 42, wherein the in vitro differentiation comprises culturing the HIP cells on feeder cells.

44. The pure population of isolated hypoimmune CAR-T cells of any one of claims 38 to 43, wherein the in vitro differentiation comprises culturing in simulated microgravity.

45. The pure population of isolated hypoimmune CAR-T cells of claim 44, wherein the culturing in simulated microgravity is for at least 72 hours.

46. The pure population of isolated hypoimmune CAR-T cells of any one of claims 38 to 42, wherein the method further comprises culturing the hypoimmune CAR-T cells in a negative selection media comprising the trigger agent to induce the HIP cells to die, thereby producing a population of isolated hypoimmune CAR-T cells that is substantially free or free of the hypoimmunogenic iPSCs.

47. A method of treating a patient with cancer by administering a composition comprising a therapeutically effective amount of the pure population of isolated hypoimmune CAR-T cells of any one of claims 38 to 46.

48. The method of claim 47, wherein the composition further comprises a therapeutically effective carrier.

49. The method of claim 47 or 48, wherein the administration comprises intravenous administration, subcutaneous administration, intranodal administration, intratumoral administration, intrathecal administration, intrapleural administration, and intraperitoneal administration.

50. The method of any one of claims 47 to 49, wherein the administration further comprise a bolus or by continuous perfusion.

51. The method of any one of claims 47 to 50, wherein the cancer is a blood cancer selected from the group consisting of leukemia, lymphoma, and myeloma.

52. The method of any one of claims 47 to 50, wherein the cancer is a solid tumor cancer or a liquid tumor cancer.

53. A method of making the isolated hypoimmune CAR-T cells of any one of claims 24 to 27 comprising in vitro differentiating of any one of the HIP cells of any one of claims 1 to 23, wherein in vitro differentiating comprises culturing the HIP cell in a culture media comprising one or more growth factors or cytokines selected from the group consisting of bFGF, EPO, Flt3L, IGF, IL-2, IL-3, IL-6, IL-7, IL-15, GM-CSF, SCF, and VEGF.

54. The method of claim 53, wherein the culture media further comprises one or more selected from the group consisting of a BMP activator, a GSK3 inhibitor, a ROCK inhibitor, a TGFβ receptor/ALK inhibitor, and a NOTCH activator.

55. The method of claim 53 or 54, wherein the in vitro differentiating comprises culturing the HIP cells on feeder cells.

56. The method of any one of claims 53 to 55, wherein the in vitro differentiating comprises culturing the HIP cells on feeder cells.

57. The method of any one of claims 53 to 56, wherein the in vitro differentiating comprises culturing in simulated microgravity.

58. The method of claim 57, wherein the culturing in simulated microgravity is for at least 72 hours.


SEQUENCE LISTING (6 sequences)

SEQ ID NO: 1 - protein, 119 aa, Homo sapiens [sequence omitted]

SEQ ID NO: 2 - protein, 160 aa, Homo sapiens
MRCLAPRPAGSYLSEPQGSSQCATMELGPLEGGYLELLNSDADPLCLYHFYDQMDLAGEEEIELYSEPDTDTINCDQFSRLLCDMEGDEETREAYANIAELDQYVFQDSQLEGLSKDIFKHIGPDEVIGESMEMPAEVGQKSQKRPFPEELPADLKHWKP

SEQ ID NO: 3 - protein, 293 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVE

SEQ ID NO: 4 - protein, 376 aa, Herpes simplex virus
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 5 - protein, 427 aa, Escherichia coli
MSNNALQTIINARLPGEEGLWQIHLQDGKISAIDAQSGVMPITENSLDAEQGLVIPPFVEPHIHLDTTQTAGQPNWNQSGTLFEGIERWAERKALLTHDDVKQRAWQTLKWQIANGIQHVRTHVDVSDATLTALKAMLEVKQEVAPWIDLQIVAFPQEGILSYPNGEALLEEALRLGADVVGAIPHFEFTREYGVESLHKTFALAQKYDRLIDVHCDEIDDEQSRFVETVAALAHHEGMGARVTASHTTAMHSYNGAYTSRLFRLLKMSGINFVANPLVNIHLQGRFDTYPKRRGITRVKEMLESGINVCFGHDDVFDPWYPLGTANMLQVLHMGLHVCQLMGYGQINDGLNLITHHSARTLNLQDYGIAAGNSANLIILPAENGFDALRRQVPVRYSVRGGKVIASTQPAQTTVYLEQPEAIDYKR

SEQ ID NO: 6 - protein, 282 aa, Homo sapiens
GFGDVGALESLRGNADLAYILSMEPCGHCLIINNVNFCRESGLRTRTGSNIDCEKLRRRFSSLHFMVEVKGDLTAKKMVLALLELAQQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS